(19) United States
(12) Patent Application Publication
(10) Pub. No.: US 2003/0149235 A1
(43) Pub. Date: Aug. 7, 2003

(54) Targeting peptides

(76) Inventors: Andrew Howard Baker, Dumbarton (GB); Stuart Anthony Nicklin, Bearsden (GB); Stephen John White, Bristol (GB)

Correspondence Address: MYERS BIGEL SIBLEY & SAJOVEC, PO BOX 37428, RALEIGH, NC, 27627, US

(21) Appl. No.: 09/990,832

(22) Filed: Nov. 16, 2001

Related U.S. Application Data
(60) Provisional application No. 60/249,702, filed on Nov. 17, 2000.

(51) Int. Cl. C07K 7/08; C07K 7/06

(52) U.S. Cl. 530/328; 530/329; 530/330

(57) ABSTRACT

Disclosed is an agent which promotes the selective uptake of material by particular cell types. In particular, disclosed are targeting peptides which promote uptake of a material by for example endothelial cells. Also disclosed is a pharmaceutical composition comprising a targeting peptide in association with a vehicle and a method of targeting a material to a cell, comprising bringing into association the targeting peptide according to the present invention with the material to be targeted to form a complex and exposing the complex to a cell(s).

### RELATED APPLICATIONS

This application claims the benefit of U.S. Provisional Patent Application Serial No. 60/249,702; Filed Nov. 17, 2000, the disclosure of which is incorporated by reference herein in its entirety.

### FIELD OF THE INVENTION

The present invention relates to peptides capable of specifically binding to particular cell types, in particular endothelial cells, and to uses of such peptides.

### BACKGROUND OF THE INVENTION

The utility of gene delivery vectors for vascular gene therapy is limited by the non-selective nature in which the vectors, either non-viral or viral, interact with the cell surface resulting in transduction of numerous cell types in addition to the target cell(s). This is further compounded by the fact that both endothelial and vascular smooth muscle cells are relatively refractory to gene delivery vectors in comparison to permissive cell types. In the context of adenoviral vectors, this is due to the low level of the Coxsackie-adenovirus receptor, CAR^{1,2} which is required for efficient virus-cell interaction. There is therefore a requirement to generate gene delivery vehicles that have an increased affinity for vascular cells.

Vascular endothelial cells are an attractive target for many gene therapeutic applications including: targeting endothelium in atherosclerosis, hypercholesterolaemia, post-angioplasty restenosis, hypertension and transplantation. Furthermore, due to the proximity of the endothelium to the bloodstream it is an attractive target for pathologies in which production of soluble proteins into the bloodstream would be a suitable gene therapy. However, uptake of viral and non-viral gene delivery vehicles by endothelial cells after intravenous or systemic delivery is low in comparison to other cells^{3,4}.

Previous studies have identified that gene delivery vectors can be targeted to individual cells types, thus generating cell-specific gene therapeutic vehicles⁵⁻¹⁰. Re-targeting adenoviruses to endothelium has been achieved with candidate ligands such as E-selectin¹¹, which targets activated endothelium in inflammatory situations, and fibroblast growth factor (FGF)^{12,13}. However, the FGF receptor is expressed on cells other than endothelium and specific targeting therefore of endothelial cells is not possible. Thus, the isolation of molecules that mediate selective and efficient attachment and internalisation of gene delivery vehicles into quiescent human endothelial cells (HEC) has not hitherto been reported.

Therefore, it is an object of the present invention to provide a reagent which promotes the selective uptake of material by particular cell types.

### SUMMARY OF THE INVENTION

Thus, in a first aspect the present invention provides a targeting peptide comprising an amino acid sequence selected from the group consisting of AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS or derivative thereof for targeting a material to a cell.

Preferably, the targeting peptide comprises an amino acid sequence selected from the group consisting of SIGYPLP, NTGPNRV, LSNFHSS, GILSPSH, MSSPGPA, MSLTTPPAVARP, MTPFPTSNEANL and MGMTVPENLIVQ, and most preferably, SIGYPLP, MSLTTPPAVARP and MTPFPTSNEANL.

Preferably the cell is a vascular endothelial cell.

These and other aspects of the present invention will become apparent from the following examples when taken in combination with the accompanying drawings, in which:

### BRIEF DESCRIPTION OF THE DRAWINGS

FIG. 1 shows modification of the S11 “adenobody” for peptide re-targeting and generation of fusion proteins. Overlapping oligonucleotides were cloned 5′ to the anti-knob single chain Fv (V_{H} and V_{K}) to create a similar spatial arrangement to that found in the phage library (i.e. peptides free at their N-terminus and constrained at their C-terminus). The PelB leader sequence is cleaved upon entry of the fusion protein into the bacterial periplasm to leave the free peptide and single chain Fv fusion protein, which can be extracted and purified;

FIG. 2 shows binding of candidate phase to different cell types. 10⁷ pfu of homogenous phage populations containing candidate peptides were panned on human endothelial cells (HEC), vascular smooth muscle cells (VSMC) and HepG2 hepatocytes, binding from a control peptide-less phage, subtracted and recovery calculated and expressed as a percentage of HEC;

FIG. 3 shows S11e inhibits adenoviral gene transfer. RAdCMV was pre-incubated for 1 hour in the presence or absence of increasing concentrations of S11e. 100 pfu/cell was then incubated with cells for 16 hours, the media changed and left for a further 48 hours. Cell extracts were quantified for the presence of beta-galactosidase.

*p<0.01 vs RAdCMV alone;

FIG. 4 shows S11eK₇ re-targets adenoviral tropism in multiple cell types. RAdCMV was pre-incubated for 1 hour in the presence or absence of S11e or S11eK₇. 100 pfu/cell was then incubated with human endothelial cells (HEC), and HepG2 hepatocytes for 16 hours, the media changed and left for a further 48 hours and stained for beta-galactosidase. The scale bar represents 250 μm and is applicable to all panels;

FIG. 5 shows S11eSIGYPLP re-targets gene transfer to human endothelial cells (HEC). RAdCMV was pre-incubated for 1 hour in the presence or absence of increasing concentrations of S11eSIGYPLP. 100 pfu/cell was then incubated with cells for 16 hours, the media changed and left for a further 48 hours. (A) X-gal staining of HEC in the absence (−, i.e. adenovirus alone) or presence (+) of increasing S11eSIGYPLP. (B) Quantification of beta-galactosidase production with increasing S11eSIGYPLP. The scale bar in (A) represents 100 μm and is applicable to all panels. *p<0.05 vs RAdCMV alone and *p<0.05 vs equivalent concentration of S11eSIGYPLP compared to HEC (n=3);

FIG. 6 illustrates the efficiency of AAV-mediated gene transfer to ECs. (A) HUVEC and HeLa cells were mock infected (open bars) or infected with 1000 transduction units/cell of wtAAVegfp (filled bars) and analyzed for eGFP expression by fluorimetry and fluorescence microscopy. (B) ECs were infected with 250 transduction units/cell wtMVegfp in the presence or absence of mg132, LLnL, genistein, hydroxyurea, aphidicolin, etoposide, or camptothecin and harvested for quantification of eGFP by fluorimetry at either 24 h or 96 h postinfection. RFU, relative fluorescent units. *Indicates statistical significance from wtMVegfp alone at the same time point. Representative of three independent experiments;

FIG. 7 illustrates the construction of an AAV mutant containing the SIGYPLP Peptide. (A) Schematic representation of AAVsig production. (B) Electron microscopy of resulting AAVwt and AAVsig virions. (C) A20 ELISA analysis and genomic titers of AAVwt and AAVsig stocks. FIG. 8 illustrates that AAVsig mediates enhanced gene transfer to EC. EC were infected with increasing doses of AAVwt or AAVsig and analyzed for beta-galactosidase expression. (A) HUVEC. (B) HSVEC. *Indicates statistical significance versus AAVwt at the equivalent dose. Representative of three independent experiments;

FIG. 8 illustrates that AAVsig mediates enhanced gene transfer to EC. EC were infected with increasing doses of AAVwt or AAVsig and analyzed for beta-galactosidase expression. (A) HUVEC. (B) HSVEC. *Indicates statistical significance versus AAVwt at the equivalent dose. Representative of three independent experiments.

FIG. 9 illustrates quantification of HSPG-independence of AAVsig. (A) Heparin-affinity column analysis of HSPG binding of AAVwt and AAVsig. We loaded 6.4×10¹¹ particles of AAVwt or AAVsig onto a prepacked and equilibrated 1 ml heparin column. Lane 1, positive control; lane 2, negative control; lane 3, MVwt or AAVsig before loading; lane 4, flow-through; lanes 5-7, sequential washing steps; lanes 8 and 9, sequential elutions with 1 M NaCl. HUVEC were infected with MVwt (B) or AAVsig (C) in the absence or presence of 1 IU heparin/5×10⁷ virions and analyzed for gene expression at 72 h. *Indicates P<0.05 versus transduction in the absence of heparin. H, heparin. Note that the scale on the y axis for (C) is 1 log greater than that for (B). Representative of three independent experiments;

FIG. 10 illustrates that AAVsig does not enhance gene transfer to nontarget cells. Human primary vascular smooth muscle cells (A) or HepG2 hepatocytes (B) were infected with low and high doses of AAVwt or AAVsig and assessed for gene expression at 72 h. *Indicates statistical significance versus AAVwt. Representative of three independent experiments;

FIG. 11 illustrates the effect of bafilomycin A2 on AAVwt and AAVsig-mediated gene expression in EC. HUVEC were infected in the presence of bafilomycin A2 (baf) and gene expression analyzed 72 h later. *Indicates statistical significance versus respective control as indicated, P<0.05. Representative of three independent experiments;

FIG. 12 illustrates the analysis of CAR-mediated transduction of Primary vascular cells. (A) Primary HUVEC, HSVEC and VSMC were infected with increasing particles/cell of AdCTL or CAR-ablated virus AdKO2G and analysed by (A) immunofluorescence (at 5000 viral particles/cell) and reporter gene expression was quantified by fluorimetry for (B) HUVEC, (C) HSVEC or (D) VSMC. *indicates P<0.05 vs AdKO2G. Representative of three independent experiments;

FIG. 13 illustrates the generation of peptide modified fibers. (A) Side and top view demonstrating structure of the Ad5 fiber (adapted from Xia et al [86]), showing the exposed surface HI loop in the fiber knob (site of peptide insertion) (B) Immunoblot analysis of recombinant fibers demonstrating efficient trimerization with either the SIGYPLP or LSNFHSS peptides incorporated into the HI loop;

FIG. 14 illustrates the production and characterisation of peptide modified fibers. (A) 293T cells are transfected with a plasmid encoding the desired fiber, and then infected 24 h later with an eGFP-expressing, fiber gene-deleted Ad vector, Ad5.GFP.ΔF. Progeny virus incorporate only the fiber encoded by the plasmid. (B) Immunoblot analysis of Ad particles with the peptide-modified fibers. Blots were probed with an anti-Ad2 fiber antibody [which cross-reacts with the Ad5 fiber (upper panel)] and then reprobed with anti-penton base protein antibody as a loading control (lower panel);

FIG. 15 illustrates the enhanced EC selectivity by SIGYPLP but not LSNFHSS fiber-modified Ad vectors. EGFP expression was quantified following infection with AdCTL, AdSIG or AdLSN. (A) Infection ratio of HUVEC relative to HepG2 hepatocytes. (B) Infection ratio of HSVEC relative to HepG2 heptocytes. (C) Flow cytometric assessment of HUVEC transduction relative to HepG2 hepatocytes. (D) Infection ratio of VSMC relative to HepG2 hepatocytes. **Indicates P<0.01 vs AdCTL. *Indicate P<0.05 vs AdCTL. AdLSN versus AdCTL, P=0.065 in VSMC; AdSIG versus AdCTL, P=0.17 in VSMC. Representative of three independent experiments. NS=not significant;

FIG. 16 illustrates the simultaneous detargeting and retargeting of adenoviral vectors. (A) Immunoblot analysis of Ad particles containing fibers with CAR-blocking mutations and SIGYPLP peptide insertion. (B-E) Quantification of reporter gene expression using fluorimetric analysis of lysates from (B) HUVEC and (C) HSVEC. (D) Western blot for eGFP in HUVEC lysates and (E) FACScan analysis for eGFP expressing HUVEC. (F) Competitive inhibition of Ad infection with soluble Ad5 fiber knob. (G) Quantification of reporter gene expression by fluorimetry in HAEC. *Indicates P<0.05 vs AdCTL. Representative of three independent experiments;

FIG. 17 illustrates the analysis of EC-selectivity mediated by AdKO1SIG. (A) VSMC and (B) HepG2 lysates were quantified for eGFP expression by fluorimetry following infection with AdCTL, AdKO1 or AdKO1SIG. (C) Ratio of reporter gene expression in AdCTL, AdKO1 SIG or AdSIG transduced EC versus HepG2 hepatocytes. *Indicates P<0.05 vs AdCTL. Representative of three independent experiments. NS=not significant;

FIG. 18 illustrates binding of candidate phage to different cell types. 10⁷ pfu of homogenous phage populations containing candidate 12mer peptides were panned on human endothelial cells (HEC), vascular smooth muscle cells (VSMC) and HepG2 hepatocytes, binding from a control peptide-less phage, subtracted and recovery calculated and expressed as a percentage of HEC;

FIG. 19 is a ClustalW multiple sequence alignment courtesy of the BCM Search Launcher; and

FIG. 20 illustrates sequence alignments of the 12mer peptides, with repeated motifs indicated by shaded boxes.

### DETAILED DESCRIPTION OF THE PREFERRED EMBODIMENTS

The material to be targeted to cells typically includes drug-delivery and gene therapy vehicles such as liposomes, viruses eg. adenovirus or adeno-associated virus vectors, bacteria, non-ionic surfactant vesicles typically known as niosomes, microcapsules, and the like. Drug delivery vehicles may generally comprise a pharmaceutically active agent, anti-tumour agents which are selectively targeted to tumour cells using targeting peptides, vaccines or the like. Examples of such vehicles are described in Watkins et al, (1997), Goldman et al (1997), Wickham et al (1996), Romanczuk et al (1999), Krasnykh et al (1998), Dmitriev et al (1998), Vigne et al (1999), Wickham et al (1995), Bartlett et al (1999), Girod et al (1999), Hall et al (1997), Kasahara et al (1994), Valsesia-Wittman et al (1994), Hart et al (1998), Hart et al (1997), Crompton et al (1994), Arap et al (1998), Eavarone et al (2000) and Tsunoda et al (1999), the contents of which are incorporated herein by reference.

The peptides disclosed herein may be used in constructs, compounds, compositions and methods in connection with other such targeting peptides or agents, including but not limited to those described in U.S. Pat. No. 6,060,316 to Young et al, U.S. Pat. No. 6,069,133 to Chiou and Carlo, U.S. Pat. No. 5,756,086 to McClelland and Stevenson, U.S. Pat. No. 6,057,155 to Wickham et al, U.S. Pat. No. 5,736,392 to Hawley-Nelson et al, U.S. Pat. No. 5,380,531 to Chakrabarti et al, U.S. Pat. No. 6,087M325 to Meers et al, U.S. Pat. No. 6,127,170 to Boutin, U.S. Pat. No. 6,071,890 to Scheule et al, U.S. Pat. No. 6,068,829 to Ruoslathi and Pasqualini and U.S. Pat. No. 6,054,312 to Larocca et al. Applicants specifically intend that the disclosures of all United States patents cited herein be incorporated by reference herein in their entirety.

The peptides of the present invention may also be associated or conjugated with agents designed to facilitate uptake into the cell such as transport peptides eg. penetratin (EP 485,578; U.S. Pat. No. 5,888,762).

Derivatives are defined as any modified forms of the protein which also substantially retains the activity of the peptides disclosed herein. Such derivatives may take the form of amino acid substitutions which may be in the form of like for like eg. a polar amino acid residue for another polar residue or like for non-like eg. substitution of a polar amino acid residue for a non-polar residue as discussed in more detail below.

Replacement amino acid residues may be selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The replacement amino acid residue may additionally be selected from unnatural amino acids. Within the above definitions of the peptides of the present invention, the specific amino acid residues of the peptide may be modified in such a manner that retains their ability to bind to endothelial cells. Thus, homologous substitution may occur i.e. like-for-like substitution such as basic for basic, acidic for acidic, polar for polar, etc. Non-homologous substitution may also occur ie. from one class of residue to another or alternatively involving the inclusion of unnatural amino acids such as ornithine (O), diaminobutyric acid (B), norleucine (N), pyriylalanine, thienylalanine, naphthylalanine and phenylglycine and the like. Within each peptide, more than one amine acid residue may be modified at a time.

As used herein, amino acids are classified according to the following classes;

basic; H,K,R

acidic; D,E

polar, A,F,G,I,L,M,P,V,W

non-polar; C,N,Q,S,T,Y,

(using the internationally accepted amino acid single letter codes)

and homologous and non-homologous substitution is defined using these classes.

Thus, homologous substitution is used to refer to a substitution from within the same class, whereas non-homologous substitution refers to a substitution from a different class or by an unnatural amino acid.

A second aspect of the present invention provides a pharmaceutical composition comprising a targeting peptide as disclosed herein in association with a vehicle, the vehicle carrying a pharmaceutically active agent; and a pharmaceutically acceptable carrier.

The pharmaceutically active agent may for example comprise a drug, a further peptide or peptides, a polynucleic acid, or the like.

The targeting peptide may be in direct association with the vehicle due for example to convalent bonding between the two, or may be “indirectly” associated, where, for example, the targeting peptide is located on or at the surface of a liposome, virus, etc. and the pharmaceutically active agent is carried by, for example contained within or associated with, the liposome, virus, etc.

It is understood that the peptide may alternatively be in direct association with the pharmaceutically active agent by, for example, covalent bonding, in the absence of a vehicle such as a vesicle.

Typically, the composition may be used to treat mammals, in particular humans.

The targeting peptides exemplified in this specification are seven and twelve amino acids long. However, a number of short motifs have been identified from sequence alignments and therefore, it is envisaged that the targeting peptides and/or derivatives thereof can be less than seven or twelve amino acids long, for example 3, 4, 5 or 6 amino acids long. They may also comprise part of a larger polypeptide chain, for example up to 15, 20, 25, 50, 75 or even 100 amino acids long, providing they still function as a targeting peptide.

In general, the term “peptide” refers to a molecular chain of amino acids with a biological activity. If required, it can be modified in vivo and/or in vitro, for example by glycosylation, myristoylation, amidation, carboxylation or phosphorylation; thus inter alia oligopeptides and polypeptides are included. It is understood however that the peptides of the present invention do not extend to native proteins which may contain the disclosed peptides. The peptides disclosed herein may be obtained, for example, by synthetic or recombinant techniques known in the art.

A further aspect of the present invention provides a method of targeting a material to a cell, said method comprising bringing into association a targeting peptide according to the present invention with the material to be targeted to form a complex and exposing the complex to a cell(s).

Preferably, the cell(s) to be targeted is an endothelial cell(s), and in particular vascular endothelial cells.

The method may be performed in vivo, for example, in gene therapy, or may be performed in vitro, for example, for use in diagnostics, transfection studies, etc. Thus, the peptides of the present invention may be also provided in a kit for use in the above described methods.

Thus, in a further embodiment, the present invention provides a kit comprising a targeting peptide comprising an amino acid sequence selected from the group consisting of AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or derivative thereof for use in transfecting or identifying cells in vitro.

The peptide may further comprise a linking region for binding to groups, such as reagents, pharmaceutically active agents, vehicles, diagnostic markers or antibodies. For example, the peptides may be modified to contain reactive groups, such as amino, carboxyl, hydroxy, thiol, phosphate, amide groups and the like known to the skilled person in the art which enable the aforementioned groups to be bound to the peptides.

In yet a further aspect, the present invention provides a method for screening targeting peptides capable of binding to an endothelial cell, said method comprising:

inserting a polynucleotide encoding a potential endothelial cell-binding peptide into an expression vector;

expressing the peptide;

conducting a pre-screening step with the expressed peptides using non-endothelial cells in order to select for the expressed peptides with reduced or negligible binding to the non-endothelial cells;

further screening the expressed peptides, which exhibited reduced or negligible binding to the non-endothelial cells, with endothelial cells; and

selecting for the expressed peptides which display selective and efficient binding to the endothelial cells.

The endothelial cells may conveniently be human endothelial cells and the non-endothelial cells may conveniently be human vascular smooth muscle cells and/or hepatocytes although other endothelial and/or non-endothelial cells may be used.

The peptides of the present invention have utility for example in gene therapy, where targeting particular nucleotide sequences or gene(s) to a particular cell-type is desirable. In an embodiment, the peptides are encoded by a nucleotide sequence which can be ligated, together with the gene(s) for targeting, to various expression-controlling DNA sequences, resulting in a so-called recombinant nucleic acid molecule. Thus, the present invention also includes an expression-controlling sequence (vector) comprising a nucleotide sequence which expresses a targeting peptide according to the present invention. Said recombinant nucleic acid molecule can then be used for transformation of a suitable host.

“Polynucleic acid” and “nucleotide sequence” as used herein refers to a polymeric form of nucleotides of any length, both to ribonucleic acid sequences and to deoxyribonucleic acid sequences. In principle, this term refers to the primary structure of the molecule, thus this term includes double stranded and single stranded DNA, as well as double and single stranded RNA, and modifications thereof.

Such recombinant nucleic acid molecules are preferably derived from for example, plasmids, or from nucleic acid sequences present in bacteriophages or viruses and are termed vector molecules.

Specific vectors which can be used to clone nucleotide sequences according to the invention are known in the art (eg. Rodriguez and Denhardt, editors, Vectors: A survey of molecular cloning vectors and their uses, Butterworths, 1988).

The methods to be used for the construction of a recombinant nucleic acid molecule according to the invention are known to those of ordinary skill in the art and are inter alia set forth in for example Sambrook et al, Molecular Cloning: A Laboratory Manual, Cold Spring Harbour Laboratory, 1989.

The present invention also relates to a transformed cell comprising the nucleic acid molecule in an expressible form. “Transformation”, as used herein, refers to the introduction of a heterologous nucleic acid sequence into a host cell in vivo, ex vivo or in vitro irrespective of the method used, for example, by calcium phosphate co-precipitation, direct uptake or transduction.

The heterologous nucleic acid sequence may be maintained through autonomous replication or alternatively may be integrated into the host's genome. The recombinant DNA molecules are preferably provided with appropriate control sequences compatible with the designated host which can regulate the expression of the inserted nucleic acid sequence.

The most widely used hosts for expression of recombinant nucleic acid molecules are bacteria, yeast, insect cells and mammalian cells. Each system has advantages and disadvantages in terms of the vector used, potential ease of production and purification of a recombinant polypeptide and authenticity of product in terms of tertiary structure, glycosylation state, biological activity and stability and will be a matter of choice for the skilled addressee.

### EXAMPLES

### Example 1

A. Materials and Methods

All chemicals unless otherwise stated were obtained from Sigma Chemical Company (Poole, UK). Cell culture reagents were obtained from Gibco BRL (Paisley, Scotland, UK) unless otherwise stated. Endothelial cell growth factor (ECGF) was from Sigma (Poole, UK). The HepG2 hepatocyte cell lines were from the European Collection of Animal Cell Cultures (Salisbury, UK). The Ph.D. Phage Display Peptide Library Kit was from New England Biolabs (Hitchin, Hertfordshire, UK) and displays random linear 7-mer peptides constrained at their C-terminus on the Pill coat protein. Von Willebrand factor (vWF) (clone F8/86) and smooth muscle cell alpha-actin (clone 1A4) antibodies were purchased from Dako (Cambridge, UK).

Cell Culture

HepG2 hepatocyte cells were maintained in Minimal Essential Media (MEM) supplemented with 100 IU/ml penicillin, 100 μg/ml streptomycin, 2 mM L-glutamine and 10% (v/v) fetal calf serum (FCS). HEC were isolated based on a modified version of the protocol described by Jaffe et al¹⁴. HEC were cultured in Dulbecco's Modified Eagle's Medium (DMEM) supplemented with 100 IU/ml penicillin, 100 μg/ml streptomycin, 2 mM L-glutamine, 20% (v/v) FCS and 1% (v/v) ECGF and used between passages 2-5. HEC were identified by immunofluorescence for von Willebrand Factor. VSMC were obtained from medial explants of human saphenous vein obtained from patients undergoing coronary artery bypass surgery⁵ and cultured in DMEM (4500 mg/L glucose) supplemented as for HepG2 cells except using 20% FCS. Peripheral blood mononuclear cells were isolated from whole blood using Ficoll Paque (Pharmacia Biotech, Uppsala, Sweden) and were cultured in RPM1640 media supplemented as for HepG2 cells.

Phage Display

Phage libraries were amplified, purified and titered according to manufacturers protocols. HEC were plated into 6 well plates and cultured until 2 days post-confluence. 1×10⁹ pfu of input phage library was used to pan confluent cultures of HEC (passage 2-4) in triplicate. First we used a direct screening approach and biopanned HEC for 4 rounds. Second we biopanned on HEC after biopanning for 1 hour on 2 successive cultures of VSMC, HepG2 and peripheral blood mononuclear cells (pre-clearing steps). Prior to biopanning growth media was changed to 1 ml DMEM containing 1% bovine serum albumin (BSA) and the plates were incubated at 4° C. for 15 minutes. For HEC, 1×10⁹ pfu of pre-cleared or stock phage library was incubated with cells for 1 hour at 4° C. Cells were washed 5 times in ice-cold PBS/1% BSA for 5 minutes per wash. Weakly associated phage were eluted in 1 ml 0.2 M glycine (pH 2.2) for 10 minutes on ice, followed by neutralisation with 200 μl Tris-HCl (pH 8.0). High affinity phage (tightly-bound phage) were isolated by lysing the cells in 1 ml of 30 mM Tris-HCL/1 mM EDTA (pH 8.0) for 1 hour on ice. Cell debris was removed and the supernatant recovered. Phage were amplified and titered between each round to ensure that 10⁹ pfu of input phage were used at the start of each successive round. Following completion of biopanning, E. coli ER2537 were infected with the resulting phage, plated and individual plaques picked, amplified, and sequenced. For further binding studies, high titer homogenous populations of each phage were prepared. Briefly, 1×10⁷ pfu of each phage were biopanned on HEC, VSMC and HepG2 cells for 30 minutes at 4° C. Unbound phage were removed by stringent washing and the resulting cell-associated phage titered. The percentage recovery was calculated for each cell type using a peptide-less phage to allow for background binding.

S11 “Adenobody” Cloning

Candidate peptides were cloned into the S11 “adenobody” re-targeting system¹⁶. We modified the S11 construct in 2 ways. First we inserted an enterokinase site at the C-terminal NotI site immediately upstream of the 6-His and myc tags using oligonucleotides (sense oligonucleotide 5′-GGCCGCAGACGACGACGACAA-3′; antisense oligonucleotide 5′-GGCCTTGTCGTCGTCGTCTGC-3′) to create pS11e (FIG. 1). We next inserted re-targeting peptides at the 5′ N-terminal end of the S11e sequence encoding the anti-fiber single chain Fv using a unique NcoI cloning site (5′-ATGGCC-3′). As a positive control we inserted the sequence encoding the peptide K-K-K-K-K-K-K using overlapping oligonucleotides (sense 5′-CATGGCCAAGGAAGAAGMAAGAAGAAGGGCGGCGGCAGCTC-3′, anti-sense 5′-CATGGAGCTGCCGCCGCCCTTCTTCTTCTTCTTCTTCTTGGC-3′ to create pS11eK₇. Since the DNA sequence encoding the peptide sequence could not be excised from the phage in order to clone it into S11, overlapping oligonucleotides were used. These are single-stranded oligonucleotides encoding the DNA sense and antisense sequences for the chosen peptides plus the necessary nucleotide overhangs to clone it into the S11 plasmid. The oligonucleotides were synthesized commercially and were ligated together in vitro so that they could be cloned into the S11 plasmid. The underlined sequence represents the peptide encoding DNA and the bold represent a tri-glycine stuffer sequence used to maintain frame and provide spacial distance from the single chain Fv. The targeting peptide S-I-G-Y-P-L-P was cloned in a similar manner but using oligonucleotides sense 5′-CATGGCCTCGATTGGGTATCCTCTTCCGGGCGGCGGCAGTC and anti-sense 5′-CATGGAGCTGCCGCCGCCCGGMGAGGATACCCAATCGAGGC-3′ to create the pS11E-SIGYPLP vector.

Recombinant Fusion Protein Production

Fusion proteins were prepared from periplasmic preparations of E. coli JM109. 500 ml of 2× TY media containing 100 μg/ml ampicillin and 0.1% glucose was inoculated with E. coli JM109 pre-transformed by standard heat-shock and plating on LB⁺ ampicillin agar plates with either pS11e, pS11eK₇ or pS11e-SIGYPLP and incubated until OD₆₀₀=0.8. Isopropyl-beta-D-thiogalactoside (IPTG) was added to 1 mM and further incubated at 30° C. for 4 hours and centrifuged at 6000 g. The pellet was re-suspended in 20 ml of PBS/1 mM EDTA, and centrifuged at 38,000 g for 30 minutes at 4° C. The resulting protein was purified on Ni-NTA columns (Qiagen, Crawley, West Sussex, UK.). Nitrilotriacetic acid (NTA) chelates metal ions such as Ni²⁺. This allows the metal ions to be immobilised on a suitable surface such as sepharose resin. This resin is then packed into a column. The nickel ions have 2 ligand binding sites free to bind tightly to stretches of histidine residues, such as those found in the 6× His tag of the S11 fusion protein. Long stretches of histidine residues are rare in prokaryotic organisms, therefore lysed bacteria can be passed down the column and only scFv with the 6× histidine tag sticks in the column. Other protein can be washed from the column, and the S11 proteins can then be eluted by washing with a high concentration of the compound imidazole. At high concentrations imadozole has a higher affinity for nickel ions than the His tag. Protein was quantified using the bicinchoninic acid (BCA) assay (Pierce, Rockford, Ill., U.S.A.) and western blotting using an anti-tetra-His antibody (Qiagen, Crawley, UK.).

Adenoviral Constructs

The adenovirus RAdCMV expresses a non-nuclear targeted LacZ gene from the cytomegalovirus immediate early promoter (CMVIEP) (see Wilkinson and Akrigg, 1992). Recombinant adenoviruses were caesium chloride-banded and titered using standard techniques.

Infection protocols and gene transfer assays. 5×10⁴ HEC, VSMC or HepG2 cells in triplicate were infected for 16 hours at 37° C. with 100 pfu/cell of RAdCMV or RAdCMV, pre-incubated for 1 hour at room temperature with between 1 and 15 μg of S11e, S11eK₇ or S11e-SIGYPLP. RAdCMV is a first-generation replication-deficient recombinant adenovirus rendered safe by deleting genes from them (in the case of first generation, this is the E1 region). The deletion of this region leaves space in the viral genome for the insertion of beta-galactosidase to allow identification of cells infected with RadCMV. The media was changed and the cells incubated for 48 hours in complete media. For histochemical analysis, cells were washed twice in sterile PBS, fixed and stained with X-Gal stain (5-bromo-4-chloro-3-indolyl-beta-D-galactopyranoside [100 mM sodium phosphate, pH 7.3 (77 mM Na₂HPO₄, 23 mM NaH₂PO₄), 1.3 mM MgCl₂, 3 mM K₃Fe(CN)₆, 3 mM K₄Fe(CN)₆ and -gal (1 mg/ml)] for 16 hours at 37° C. Cells were washed in PBS and the nuclei counter-stained with Mayer's haematoxylin, dehydrated and mounted in DPX (BDH, Poole, Dorset, UK.). beta-galactosidase was quantified using a chemiluminescent assay (Galacto-Light Plus, Tropix, Mass. U.S.A.). Briefly, 48 hours post-infection cells were lysed for 10 minutes at 4° C. in 50 μl of lysis buffer. 2 μl of lysate was added to 200 μl of reaction buffer and incubated for 1 hour at room temperature. 300 μl of reaction accelerator was added and samples were assayed in a Biorbit luminometer (Life Sciences, Basingstoke, Hampshire, UK). The mean was calculated over a 5-second period. Each sample was quantified using a standard curve.

Statistical Analysis

All data were analysed using unpaired Students' t-test and are shown as mean value “the standard error of the mean (SEM). Data were considered significant when p<0.05. All experiments were performed in triplicate and repeated on at least a further 2 independent occasions.

B. Results

Phage Display Identifies Peptides that Target HEC

The Ph.D.™ ligand screening system is based on a combinatorial library of random peptide 7-mers fused to a minor coat protein (pIII; 8 copies per phage) of the filamentous coliphage M13. Phage display creates a physical linkage between a selectable function (the displayed peptide sequence) and the DNA encoding that function. This allows rapid identification of peptide ligands for a variety of receptor target molecules (antibodies, enzymes, etc) by an in vitro or in vivo selection process called biopanning. Biopanning is carried out by passing the pool of phage-displayed peptides over a plate (or bead) coated with the target, washing away the unbound phage, and eluting the bound phage. The eluted phage is then amplified and taken through additional binding/amplification cycles to enrich the pool in favour of binding sequences. After 3-4 rounds, individual clones are characterised by DNA sequencing.

We designed 2 independent strategies to isolate endothelial cell-targeting phage. First, we exposed post-confluent cultures of HEC to the phage library and completed 4 rounds of panning to identify peptide sequences. To define that we were selectively enriching the library for endothelial-targeting phage after each round of biopanning, we titered phage (Table 1).

TABLE 1
Recovery of phage from human umbilical vein endothelial cells (HEC).

| Round | Weakly Associated Phage | Tightly Bound Phage |
|---|---|---|
| 1 | 1.6″0.2 × 10⁴ | 3.4″0.8 × 10⁴ |
| 2 | *2.3″0.4 × 10⁴ | *6.9″0.9 × 10⁴ |
| 3 | *3.2″0.3 × 10⁵ | *4.1″0.7 × 10⁵ |
| 4 | 1.7″1.3 × 10⁶ | *2.1″0.2 × 10⁶ |

Following each round of panning on HEC, phage were titered. *p < 0.05 vs titer recovered from the previous round of panning (n = 3). Only tightly-bound phage were selected for further experiments.

A significant increase in the titer of phage was observed (Table 1). Following four rounds we isolated and sequenced 60 individual phage (Table 2).

58 different peptide sequences were identified, one peptide (LTAELTP) appearing twice and a further phage contained no inserted peptide sequence (Table 2). A number of motifs were identified. For example, the peptides IVAQPRL, QPRLLHH, and TRSQPPL all contain the sequence QP(P/R)L (Table 2). Other examples include two further peptides, INSNAPG and INSVSPH which contain the motif INS at the N-terminus (Table 2).

TABLE 2
Peptide seguences obtained from panning
human umbilical vein endothelial cells (HEC).

| | Fre- | | Fre- | | Fre- |
|---|---|---|---|---|---|
| Peptide | quency | Peptide | quency | Peptide | quency |
| AASARLP | 1 | VYFPAPN | 1 | FSMSTPS | 1 |
| IVAQPRL | 1 | FPQTYTT | 1 | NIAAFSL | 1 |
| QPRLLHH | 1 | NIIPAPT | 1 | SPTYPRR | 1 |
| TRSQPPL | 1 | NTGPNRV | 1 | PPPDWTF | 1 |
| SHFSHLR | 1 | AFNYPPH | 1 | DFLQVSP | 1 |
| SPDHLFC | 1 | LEHPPTT | 1 | TYPSSEW | 1 |
| IPMHLHN | 1 | TSESPTV | 1 | YSLSRSL | 1 |
| NHLSALY | 1 | TYSLKSA | 1 | TSTMPSR | 1 |
| ETIKTNT | 1 | ATGFATP | 1 | TNSQPSP | 1 |
| TSFFMPP | 1 | TAAYRFW | 1 | LPPSLYS | 1 |
| SPSVVPF | 1 | HSLTFSI | 1 | WNSTTQA | 1 |
| HFTHPTH | 1 | AGATAMS | 1 | STYPIIR | 1 |
| SWNHARV | 1 | NHWHGGL | 1 | GILSPSH | 1 |
| EAVPTYS | 1 | INSNAPG | 1 | YSTHSTR | 1 |
| SDLATVR | 1 | INSVSPH | 1 | MSSPGPA | 1 |
| LPTKTLF | 1 | AAWPTSS | 1 | LTAELTP | 2 |
| KIDGTPR | 1 | VEPARAS | 1 | SIGYPLP | 1 (45) |
| WTSDELH | 1 | TLGLHMS | 1 | LSNFHSS | 1 (2) |
| SLPRNSD | 1 | GYQQVFQ | 1 | MSPPGPA | 0 (33) |
| LCMTTLV | 1 | SEVAVQG | 1 | NO INSERT | 1 |

Peptide sequences and their frequencies obtained from phage display using direct panning on HEC and biopanning following pre-clearing (figures in parentheses).

We next modified biopanning to include pre-clearing on successive cultures of VSMC, hepatocytes and peripheral blood mononuclear cells prior to biopanning on HEC. A further 80 individual phage were sequenced (Table 2). Using this strategy only 3 different peptides were isolated. The peptides SIGYPLP and MSPPGPA and LSNFHSS appeared 45, 33 and 2 times respectively. Additionally the peptides SIGYPLP and LSNFHSS appeared by both screening approaches while MSSPGPA from the direct screening approach differed by only one amino acid from MSPPGPA isolated in the pre-clearing strategy. For all peptides that appeared more than once, the DNA coding sequences were identical (data not shown).

Pre-Selection of Peptides from Phage Display Identifies Candidates for Re-Targeting

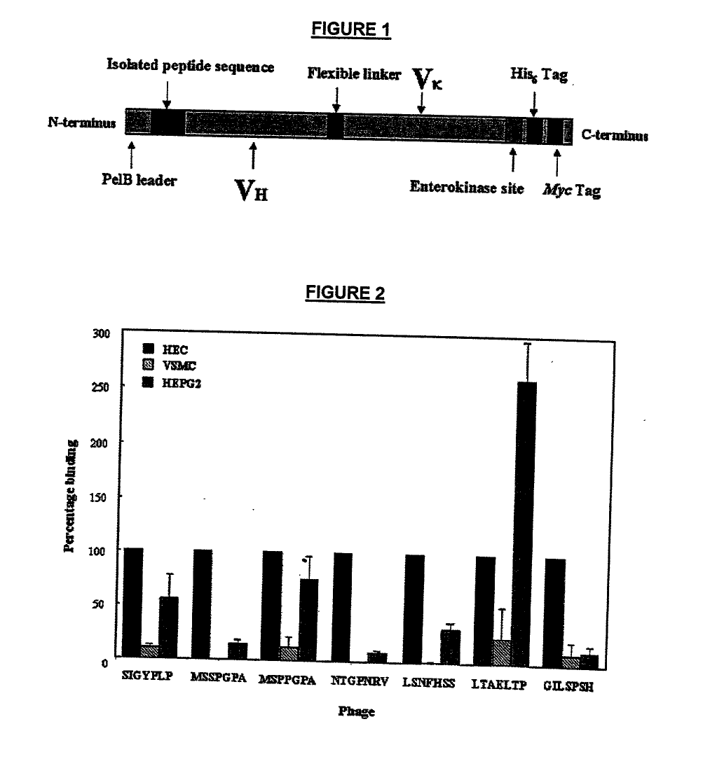

Following selection of putative endothelial-specific peptides, pure high-titer stocks of homogenous phage were generated for further characterisation of their binding characteristics to HEC, VSMC and HepG2 cells (FIG. 2). Recovery of phage from VSMC varied from between 0% for NTGPNRV to 24.1″27.8% for LTAELTP compared to the recovery from HEC and all 7 peptides tested were lower in VSMC than HEC. Diversity was observed in the recovery of phage from HepG2s, varying from 8.9″1.8% for NTGPNRV to 260.0″36.0% for LTAELTP compared to HEC (FIG. 2). We therefore selected the peptide SIGYPLP for further investigation due to its consistent appearance in biopanning and its limited binding to non-endothelial cell types compared to HEC.

Modification of the S11e “Adenobody” Single Chain Fv Re-Targets Adenoviral Tropism Defined by 7-mer Peptides

The S11 adenobody re-targeting system is based on a single chain Fragment variable (scFv), the smallest part of an antibody which retains its specificity for an antigen. In the case of S11, the scFv binds to the knob protein of the adenovirus thereby preventing it from binding to its primary cell-surface receptor, the coxsackie/adenovirus receptor (CAR). The DNA sequence for this antibody has been cloned into a plasmid vector. This enables it to be engineered by cloning additional sequences upstream or downstream of the scFv (eg. DNA encoding peptide sequences). In this way the joined DNA sequences form a fusion protein when the plasmid is introduced into bacteria and grown at 37EC. See Watkins et al (1997) Gene Therapy 4: 1004-1012.

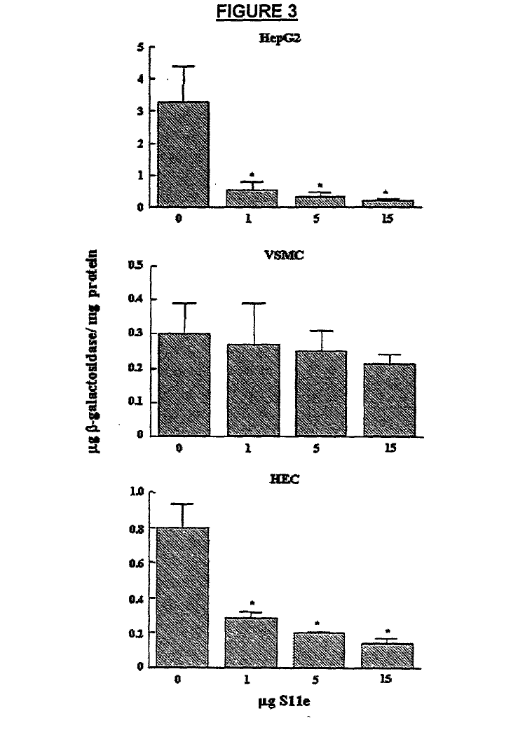

We first modified the S11e “adenobody” 16 to enable cloning of short peptides to create fusion proteins (FIG. 1). We designed the cloning so that the peptides were fused to the N-terminus of the single chain Fv and were therefore constrained at the C-terminus (identical to the peptide presentation within the phage library). The enterokinase tag was inserted into the S11 adenobody to allow the His and myc tags to be cleaved if the extra protein sequence interfered with the endothelial cell targeting. However, this was not necessary with the SIGYPLP peptide. We next defined that the S11e protein (without a re-targeting peptide) could block fibre-dependent entry of RAdCMV into different cell types (FIG. 3). Pre-incubation of RAdCMV with S11e produced a dose-dependent reduction in the level of beta-galactosidase expression from HepG2 cells (FIG. 3, p<0.05, n=3)¹⁶. Different results were obtained with VSMC and HEC. In the absence of S11e, RAdCMV infection into both VSMC and HEC was much lower than that observed for HepG2 cells (FIG. 3). For VSMC, there was no significant reduction in beta-galactosidase production in the presence of increasing concentrations of S11e, presumably due to the low level of fiber-dependent entry of adenovirus into VSMC (FIG. 3). Infection into HEC, was however, significantly inhibited by increasing concentrations of S11e (FIG. 3, p<0.05, n=3).

Inclusion of a Poly-Lysine Peptide at the N-Terminus of S11e Re-Targets Adenoviral Infection

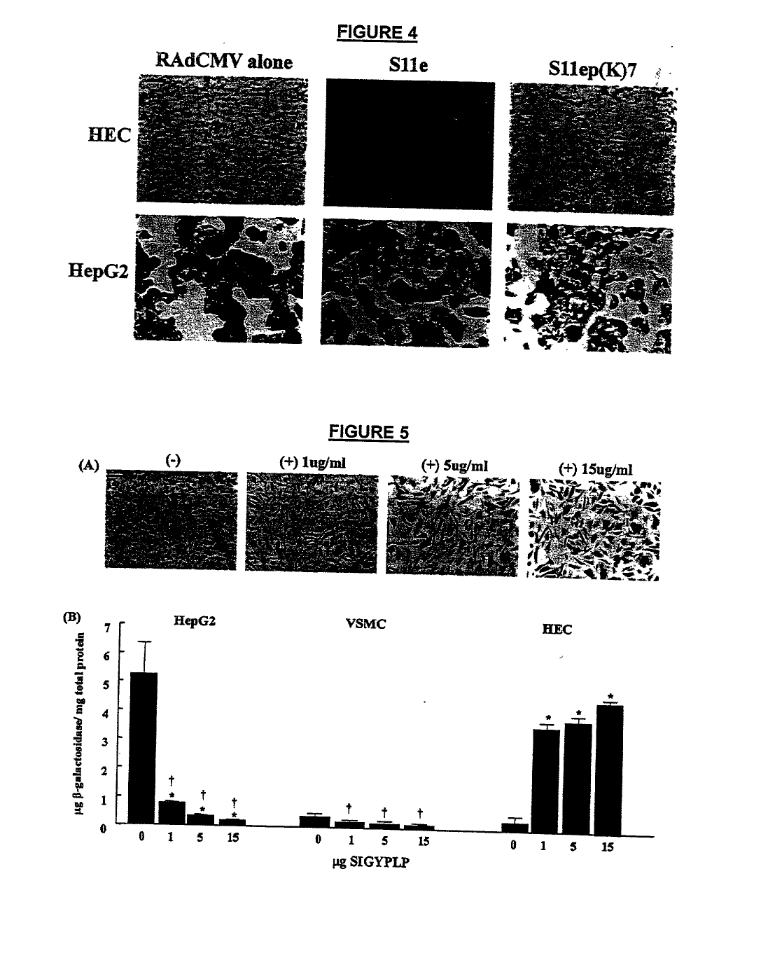

We next cloned overlapping oligonucleotides encoding the peptide sequence KKKKKKK into S11e to create S11eK₇ and purified the fusion protein. We observed that the S11eK₇ re-defined adenoviral tropism, with an increase in infection of both HepG2 and HEC compared to cells incubated with S11e alone (FIG. 4). In fact, for HEC the level of transduction achieved with S11eK₇ was above that observed using RAdCMV alone demonstrating the inefficiency of adenoviral entry into HEC (FIG. 4).

The Peptide SIGYPLP Re-Targets Gene Transfer Selectively to Endothelial Cells

Following cloning of the SIGYPLP peptide into S11e to create S11eSIGYPLP, we analysed the ability of this peptide, isolated from the phage library, to re-direct gene transfer to HEC. S11eSIGYPLP induced an identical pattern of beta-galactosidase expression in non-endothelial cells (VSMC and HepG2 cells) to S11e alone. However, S11eSIGYPLP caused a significant increase in HEC cell transduction compared to the level achieved with S11e or RAdCMV alone (FIG. 5, p<0.01, n=3). Furthermore, the level of beta-galactosidase expression increased from 0.29″0.2 μg beta-galactosidase for infection with adenovirus alone to 4.52″0.11 μg beta-galactosidase/mg protein for SIGYPLP-mediated infection representing a 15.5 fold increase in transduction efficiency and resulting in transduction levels similar to that seen for normal non-targeted adenoviral infection into HepG2 hepatocyes (FIG. 5, p<0.05, n=3).

C. Discussion

Due to the inability of current gene delivery systems to efficiently transduce endothelial cells in comparison to other cell types, we have sought to identify small, novel peptide sequences that can be incorporated into gene transfer vectors in order to mediate selective and efficient vehicle-to-endothelial cell interactions. For this purpose, we utilised random phage display libraries expressing 7-mer peptides on their coat proteins to pan primary, post-confluent cultures of endothelial cells. We identified 59 peptides using 2 independent panning strategies and further characterised the binding of 7 peptides to HEC, VSMC and HepG2 hepatocytes. The peptide SIGYPLP, which was isolated by both panning procedures, demonstrated high-level binding to HEC but low-level affinity for both VSMC and hepatocytes, and was therefore cloned into the S11e adenoviral system. We discovered that inclusion of SIGYPLP not only targeted endothelial cells in a cell-specific manner, but also increased transgene expression significantly higher than that achieved with non-targeted adenovirus.

Phage display has previously been demonstrated as a feasible strategy for obtaining small peptides that facilitate protein interactions¹⁸⁻²². We included a pre-clearing step to increase the efficiency of the biopanning by removal of peptides which could mediate interactions with ubiquitously expressed cellular receptors. Indeed, pre-clearing enriched for candidate peptides as the phage containing peptide SIGYPLP appeared only once without pre-clearing, but 45 times with pre-clearing. The success of the technique was highlighted when high-titer stocks of pure populations of these peptide expressing phage were used in repeated biopanning experiments on HEC, HepG2 and VSMC. Here, all 7 peptides tested showed significantly higher recovery from HEC than VSMC.

The wide tropism of adenovirus is a disadvantage in many clinically applicable situations for gene therapy when tissue restricted gene expression is required. Thus, the development of suitable re-targeting systems for adenovirus has been the goal for many researchers. Systems developed for re-targeting adenoviral vectors have included non-genetic modifications of the adenoviral fiber by the use of Fab fragments of antibodies chemically conjugated to re-targeting ligands^{12,13,23}, bi-specific antibodies^{6,7,11}, with primary specificity to a component of the adenoviral fiber and a secondary specificity for a cell-associated protein, or by the use of scFv fragments¹⁶, genetically fused to re-targeting ligands. In our system we observed a large enhancement of infection using SIGYPLP. This observation can only improve the safety profile for gene transfer when used to target gene expression from endothelial cells.

It has previously been demonstrated that small peptide motifs are able to mediate specific cellular interactions, e.g. integrins with RGD motifs^{24,25}. Previous studies utilising the method of in vivo phage display have also demonstrated that phage expressing small peptides are able to home to specific vascular beds^{26,28} and act as drug delivery agents to specific cell types²⁹. However, although candidate integrin targeting strategies using RGD motifs have been demonstrated to enhance adenoviral infection generally^{6,30} a novel linear peptide obtained by panning a random library has not.

### Example 2

Adenoviral vectors have demonstrated proof-of-concept in diverse preclinical studies in cardiovascular disease. However, for development of gene therapy for clinical cardiovascular applications, immune responses to Ad vectors in the vessel wall [48] may preclude their use, even with the advent of second- or third-generation vectors. In studies using genetic models of human essential hypertension, AAV vector systems have shown efficacy [49, 50], but AAV vectors demonstrate relatively poor infectivity for EC in comparison with other cell types, even when administered locally [51, 52]. AAV2 tropism is defined by binding to heparan sulphate proteoglycans [53] and the use of alphaᵥbeta₅ integrins or basic fibroblast growth factor receptor (FGFR1) as coreceptors [54, 55]. Recent studies have demonstrated incorporation of foreign epitopes into defined sites of the AAV2 capsid [32, 56]. Incorporated peptide motifs did not disrupt capsid integrity and enabled binding to new receptors. But, so far, modification of AAV for targeting to single selected cell populations has not been described. We have therefore sought to genetically modify AAV tropism to increase transduction of vascular EC in an efficient and selective manner using a 7-mer peptide, SIGYPLP, originally isolated by phage display [47]. We report here the construction and evaluation of a novel AAV vector genetically modified with SIGYPLP and demonstrate efficient and enhanced AAV-mediated gene delivery to vascular EC.

A. Materials and Methods

Cell Culture

Primary human umbilical vein endothelial cells (HUVEC) were purchased from TCS Cellworks (Botolph Clayden, Bucks, UK), cultured according to the manufacturer's recommendations and used below passage 5. Primary human saphenous vein endothelial cells (HSVEC) were prepared by collagenase digestion of human saphenous veins obtained from patients undergoing coronary artery bypass graft surgery (CABG) and were cultured as for HUVEC. Primary human saphenous vein smooth muscle cells (SMC) were prepared from excess saphenous veins of CABG patients using the explant technique [15]. Cells were cultured in Dulbecco's modified Eagle's medium containing 20% fetal calf serum (FCS), 100 μg/ml penicillin, and 100 IU/ml streptomycin. All cells were maintained at 37° C. with 5% CO₂. HepG2 hepatocyte and 293 cells (from ATCC) were maintained as for SMC except in the presence of 10% FCS.

Plasmid Construction

pZnL was constructed by inserting the BgiII fragment of pADLacZ (plasmid obtained from Axel Doeniecke) into the SnaBI site of psub201(+) [69] (plasmid obtained from R. J. Samulski) producing an AAV2-based vector with the zeocin selectable marker and Escherichia coli gene lacZ with a nuclear localization signal downstream of the cytomegalovirus immediate early promoter and flanked by AAV2 inverted terminal repeats (ITRs). pRCwt is a helper plasmid for virus construction encoding the wild-type AAV capsid and was constructed by blunt-end subcloning of the 4.5-kb XbaI fragment of psub201(+) into the PstI and BamHI site of pSV40OriAAV [70]. PRCsig (helper plasmid for packaging SIGYPLP peptide) was constructed using overlapping oligonucleotides. Oligonucleotides (sense, 5′-CGCGTCGATCGGTTACCCATTGCCAG-3′; antisense, 5′-CGCGCTGGCAATGGGTAACCGATCGA-3′) encoding SIGYPLP were annealed and inserted into the MluI-AscI site of pRC'99, thus incorporating the peptide sequence at position 1-587 of the AAV2 capsid [32]. PRC'99 was generated by inserting the overlapping sequence of the restriction enzymes MluI, BspEI, and AscI into the amino acid position 1-587 of the AAV2 capsid cloned in pRCwt. The pXX6 adenovirus helper plasmid [71] was provided by R. J. Samulski.

Packaging of AAV with Wild-Type and SIGYPLP-Modified Capsids

The eGFP-expressing AAV vector wtAAVegfp has been described [72]. For preparation of retargeted AAV vectors, 15 plates of 293 cells at 80% confluence were cotransfected with 37.5 μg of pZnL and pRCwt (wild-type capsid) or pRCsig (mutant capsid) and pXX6 at a 1:1:1 molar ratio. After 48 h cells were collected and pelleted by centrifugation. Cells were resuspended in 150 mM NaCl, 50 mM Tris-HCl (pH 8.5), freeze-thawed several times, and treated with Benzonase (50 U/ml) for 30 min at 37° C. [73]. Cell debris was removed by centrifugation, supernatant loaded onto an iodixanol gradient as described [73] and subjected to centrifugation at 69,000 rpm for 1 h at 18° C., and the resulting 40% iodixanol phase containing AAV harvested.

Evaluation of AAV Titers

We evaluated AAVwt and AAVsig preparations for particle titer by ELISA and genomic titer by dot-blot analysis. Briefly, for ELISA (Progen, Heidelberg, Germany) serial dilutions of AAV were prepared in sample buffer, plated onto A20 coated microtiter plates, and incubated for 1 h at 37° C. Following detection at 450 nm, capsid titers were quantified according to the manufacturer's recommendations using an internal AAV-positive control. Genomic titers were quantified by dot-blot analysis as described [32]. Serial twofold dilutions of AAV preparations were denatured in 2 M NaOH, slot blotted and dried on nylon membranes (Boehringer, Germany), and hybridized using a DIG-labelled LacZ probe. Transducing particle titers of retargeted AAV vectors were determined by infecting 2×10⁴ HeLa cells with serial twofold dilutions of the AAV preparations in the presence of adenovirus type 5 (multiplicity of infection of 1). After 72 h, the cells were stained in situ using the X-gal assay [70] and the transducing viral particle titer evaluated. Signals were normalized to an internal standard. AAVwtegfp transducing unit titers were determined essentially as above, except, at the required time point, eGFP-positive cells were counted under fluorescence microscopy.

Electron Microscopy

Iodixanol gradient-purified viral particles were adsorbed onto Formvar-carbon-coated copper grids, negatively stained with uranyl acetate, and analyzed by transmission electron microscopy (DKFZ, Heidelberg, Germany).

AAV Infection Assays

Cells were plated to reach 80% confluence 24 h later. In each case an accurate cell count was performed immediately before infection. Cells were infected with increasing doses of AAV vectors in the presence or absence of helper adenovirus (Ad) as indicated. For each cell type, Ad co-infection was performed at a dose that mediated 70% transduction, based on prior infection efficiency experiments performed on each cell type using a LacZ-expressing Ad, RAd35. At the required time point, cells were washed and lysed in 0.2% Triton X-100 in PBS and stored at −20° C. For dissection of AAV transduction of EC, cells were incubated with AAV in the absence or presence of 40 μM LLnL, 4 μM mg132, 50 μM genistein, 40 mM hydroxyurea, 5 μg/ml aphidicolin, 10 μM etoposide, 0.1 μM camptothecin or 150 nM bafilomycin A₂.

Heparin Competition

Cells were infected as indicated above in the absence or presence of 1 IU heparin/5×10⁻⁵ genomic particles and analyzed for gene expression 72 h later. In addition a column assay was carried out [56]. AAVwt or AAVsig were loaded onto heparin columns (immobilized on cross-linked 4% beaded agarose; Sigma) that had been prepacked and equilibrated according to the manufacturer's recommendations. Purified viral preparations (6.4×10¹¹ particles) in 200 μl were loaded, and the flow-through, wash, and 1 M NaCl eluates were collected and analyzed by dot-blot hybridization using twofold serial dilutions. For quantification of wtAAVegfp-mediated gene transfer to vascular EC, aliquots of cell extracts were analyzed in triplicate for eGFP expression using a Wallac Victor2 multi-counter with recombinant eGFP (Clontech, Basingstoke, Hants., UK) as a standard. All data were analyzed as eGFP relative light units (RFU)/mg protein. For analysis of AAVsig and AAVwtdriven lacZ expression, aliquots of cell extracts were analyzed for beta-galactosidase expression using Galacto-Light Plus (Tropix, Mass., U.S.A.) as per manufacturer's recommendations using recombinant beta-galactosidase (Sigma, UK) as a standard. All data were expressed as RLU/mg protein.

Statistics

All data were analyzed between groups using Student's unpaired t test and were considered significant when P<0.05.

B. Results

Inefficient AAV-Mediated Gene Transfer to Vascular EC

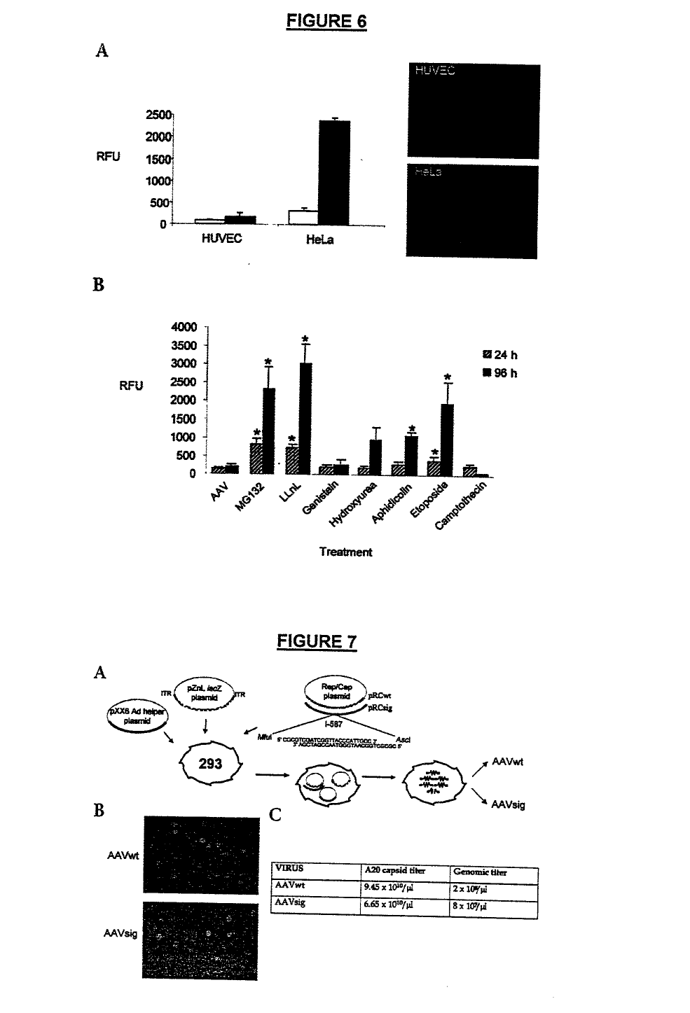

Previous studies have demonstrated that non-modified AAV vectors can transduce vascular endothelial cells [52, 57]. However, the relative infectivity of AAV for EC compared with other cell types (such as vascular smooth muscle cells, immortalized cell lines, skeletal muscle, neurons, or hepatocytes) is low [43,44,45,46,51,57]. Lack of efficient EC transduction has been demonstrated both by local delivery to the vessel wall [52] and systemically by the sequestration of AAV vectors into organs such as the liver and spleen following intravenous infusion [58,59]. The reason for this inefficiency is not known, although it may reflect inefficient AAV tropism for EC combined with inefficient nuclear trafficking of internalized AAV particles, as has been described in other cell types [60-62]. Using an eGFP-expressing AAV (wtAAVegfp), we initially compared AAV-mediated transduction of EC and AAV-permissive HeLa cells, in vitro. Infection of human umbilical vein endothelial cells (HUVEC) with 1000 transduction units per cell (TU/cell) of wtAAVegfp resulted in a very low level of reporter gene expression four days postinfection, confirming the relative inefficiency of AAV-mediated gene transfer to EC (FIG. 6A). Conversely, HeLa cells demonstrated high-level reporter gene expression using an identical TU/cell (FIG. 6A). We next investigated whether the observed inefficient EC transduction was a result of poor nuclear trafficking of internalized AAV by characterizing wtMVegfp transduction in the presence of agents that affect components of the AAV intracellular transduction pathway, including proteasome inhibitors, genotoxic agents, and topoisomerase inhibitors (FIG. 6B). Infection with wtMVegfp in the presence of the proteasome inhibitors LLnL or mg132 evoked an increase in reporter gene expression in comparison with infection without treatment, confirming that AAV particles are targeted for proteasomal degradation in EC (FIG. 6B). Similar to other cell types [62], reporter gene expression in both HUVEC (FIG. 6B) and human saphenous vein endothelial cells (HSVEC; data not shown) was also enhanced in the presence of aphidicolin and etoposide.

Generation of a Mutant AAV with the SIGYPLP Peptide Incorporated into the Capsid

Based on these findings, we sought to engineer a retargeted recombinant AAV vector that would mediate enhanced infection into EC. We previously demonstrated that insertion of small peptides at position 1-587 in the AAV2 capsid are well tolerated and mediate targeting to the receptor defined by the inserted epitope [32]. We therefore incorporated the EC-targeting peptide, SIGYPLP, a 7-mer peptide isolated from a linear phage display library [47], into position 1-587 of the AAV capsid (FIG. 7A), creating AAVsig. Incorporation of SIGYPLP resulted in production of virions with intact capsids as assessed by electron microscopy (FIG. 7B) and A20 ELISA (FIG. 7C). Genomic particle titrations were similar to that achieved for AAVwt (FIG. 7C), thus SIGYPLP insertion had no deleterious effect on AAV capsid integrity and particle production.

SIGYPLP Enhances AAV-Mediated Transduction of Primary EC

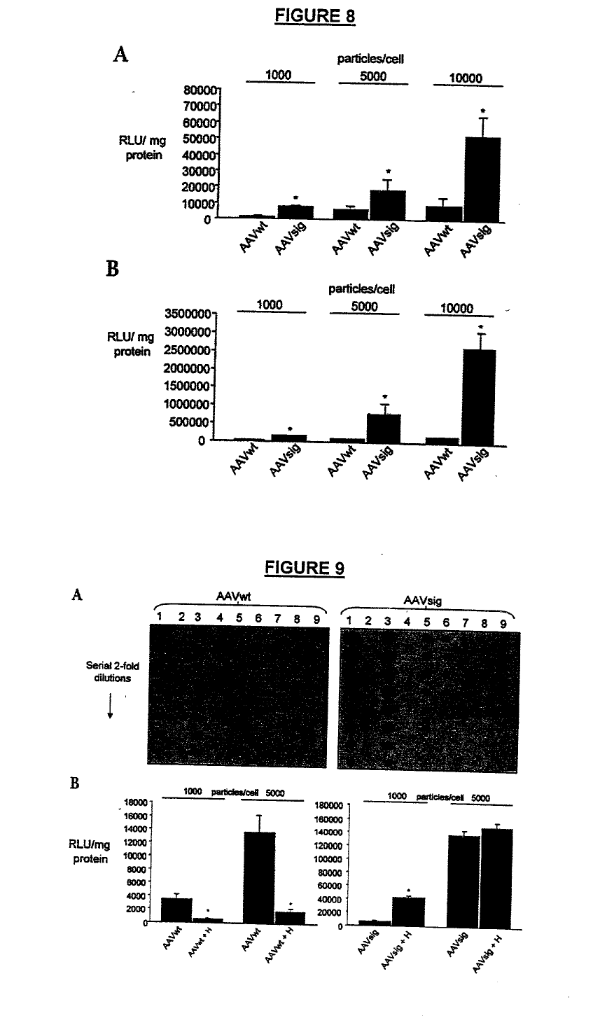

The SIGYPLP EC-targeting peptide was originally isolated by phage display as a linear peptide fused to the bacteriophage M13 pill protein and therefore constrained only at its carboxy terminus [47]. Targeting fidelity was maintained when the peptide was engineered in an identical spatial configuration into bi-specific antibodies and used to retarget adenoviral vectors [47]. As genetic incorporation of SIGYPLP into AAV capsids results in its constraint at both amino and carboxy termini, we first sought to determine if AAVsig could maintain its EC targeting capacity and then compared levels of reporter gene expression with those produced by AAVwt. HUVEC and HSVEC cells were exposed to increasing particle doses of MVsig or AAVwt and analyzed for transgene expression after 72 hours (FIG. 8). As expected, for both AAVwt and MVsig, dose-dependent increases in gene expression were observed with increasing particle titers. However, AAVsig evoked significantly higher transgene expression than AAVwt in both HUVEC (FIG. 8A) and HSVEC (FIG. 8B). The increase in gene expression was 5.9-fold for HUVEC and 28.2-fold for HSVEC at 10,000 particles per cell. AAVsig-induced gene expression in HSVEC was higher than in HUVEC, perhaps reflective of variation in the levels of the as-yet unidentified receptor for SIGYPLP in EC from different vascular beds. Thus, SIGYPLP maintained its targeting fidelity when fully constrained within the AAV capsid.

AAVsig Transduces EC Independently of HSPG Binding

For definitive retargeting of AAV to individual cells, peptides that mediate binding to cell surface receptors independently of HSPG binding are the first requirement. To address the HSPG binding capacity of AAVwt and AAVsig, we first used heparin affinity columns. Loading of AAVwt preparations onto 1 ml heparin columns resulted in efficient retention of particles in the column with very little detected in the column wash (FIG. 9A). Following elution with 1 M NaCl, we recovered AAVwt virions (FIG. 9A). In contrast AAVsig was not retained in the heparin column, but was detected in the column wash and not in the 1 M NaCl eluate (FIG. 9A). To evaluate the dependence of AAV-HSPG binding for transduction, we infected EC with AAVwt or AAVsig in the presence or absence of competing heparin and quantified reporter gene expression (FIG. 9B). While gene expression from AAVwt was almost totally abolished in infected vascular EC, gene expression from AAVsig was not reduced, demonstrating that transduction of EC with AAVsig was independent of binding to HSPGs.

AAVsig Does Not Transduce Human SMC or Hepatocytes

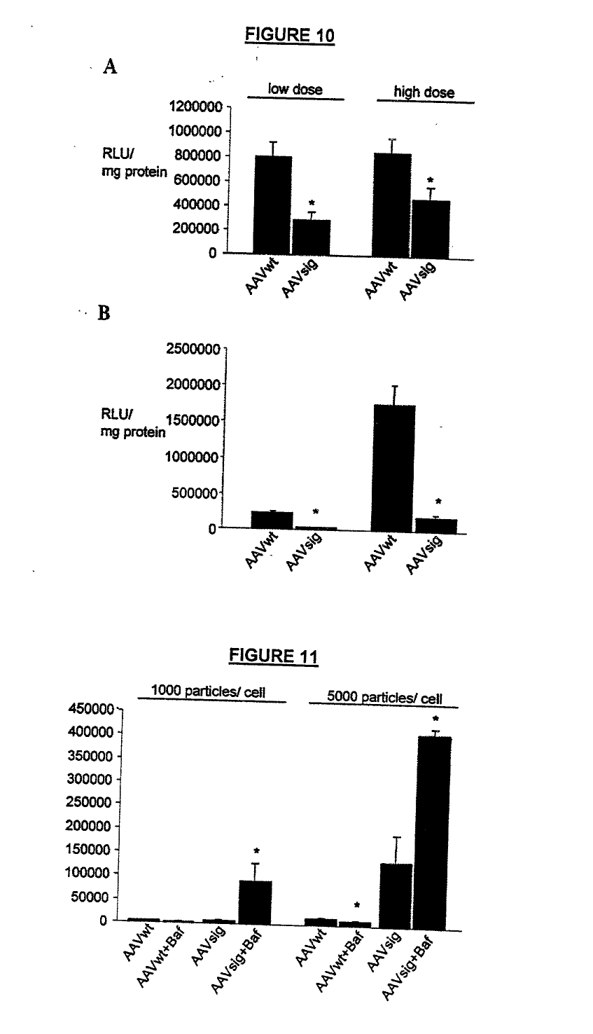

Safety and efficacy of retargeted AAV vectors would be further enhanced by selectivity in addition to enhanced tropism for the desired cell type. Following local delivery applications to blood vessels, smooth muscle cells (SMC) in the vessel media are the predominant cell type infected [51] and similarly, for systemic applications, AAV vectors are sequestered in organs such as the liver, where they infect hepatocytes [59]. We therefore evaluated whether SIGYPLP transduced SMC and HepG2 hepatocytes (FIGS. 10A and 10B). In direct contrast to reporter gene expression produced in primary EC, AAVsig evoked significantly lower reporter gene expression than AAVwt in both SMC and HepG2 hepatocytes (FIGS. 10A and 10B). Therefore, in addition to enhancement of EC transduction, SIGYPLPmodified AAV demonstrated relevant selectivity.

SIGYPLP Alters Intracellular Trafficking of AAV in EC

As AAVsig has a distinctly modified tropism compared with AAVwt, transduction of EC with AAVsig may involve different intracellular trafficking pathways in comparison with AAVwt, whose pathway is defined by routing through late endosomes [60,61]. We therefore assessed the action of agents that affect endosome trafficking and maturation and proteasome activity on AAVsig-mediated gene transfer in comparison with AAVwt. Similarly to previous studies [61,62], incubation with bafilomycin A2 (an inhibitor of endosomal acidification) reduced AAVwt-mediated gene transfer (FIG. 11). Conversely, for AAVsig-mediated gene transfer, bafilomycin A2 substantially enhanced transduction at all doses examined (FIG. 11).

C. Discussion

For AAV-mediated gene transfer to be applicable for diseases for which efficient and selective gene delivery to vascular endothelial cells is a prerequisite, novel EC-targeting AAV vectors are required. Here we have focused on the relative inefficiency of AAV-mediated gene transfer to EC and on the development and characterization of a novel genetically engineered EC-targeted recombinant AAV vector. We have shown the ability of the EC-homing peptide SIGYPLP to incorporate efficiently into AAV virions and to mediate enhanced, targeted gene transfer to EC. Detailed mapping of AAV capsid proteins [63,64] has shed light on the mechanisms by which AAV particles interact with target cells and has identified domains that allow foreign epitope insertion [32,56]. In a previous study [56], panels of AAV mutants were generated by incorporation of foreign epitope tags, insertion of ligands, alanine scanning mutagenesis, and amino acid substitutions. A number of phenotypes were produced in the resulting AAV vectors, enabling the pinpointing of mutations that maintained efficient capsid assembly and A20 ELISA positivity and allowed evaluation of HSPG binding capacity. Previous attempts to retarget AAV vectors have reported mixed results, illustrating the potential technical barriers for achieving effective and selective retargeting. Yang et al. [65] fused a gene encoding a single chain antibody against CD34 to VP1, VP2, and VP3 genes and demonstrated that a VP2-CD34 fusion construct produced infectious recombinant AAV particles that were able to elevate transduction of KG-1 cells, which express the CD34 receptor. However, this method packaged AAV progeny in the presence of both wild-type VP2 and modified VP2, thus producing heterogeneous viral preparations consisting of both retargeted and wild-type AAV capsids [65]. Although a proof of principle, the coproduction of wild-type and retargeted virions, combined with the low infectious titers generated, precludes the use of this strategy as an efficient AAV retargeting system. Bi-specific antibody approaches have also been used as a retargeting strategy. An antibody with dual specificity for the AAV capsid and the cell surface alpha_{IIb}beta₃ integrin, which is expressed on megakaryocytes and platelets, demonstrated efficient retargeting to DAMI and MO7e cells [66]. Bi-specific strategies such as these have been used successfully for adenovirus-based retargeting to EC, with high efficiency both in vitro and in vivo [47,67,6]. However, two component systems raise issues over production scale-up, affinity of the individual bi-specific antibodies for each component and the stability of the complexes in vivo. Therefore, the development of single-component, genetic based AAV retargeting is the preferred strategy.

In comparison with retroviruses and adenoviruses, relatively little is known about the structure, conformation, and characteristics of AAV coat proteins. This, however, has not hindered successful genetic retargeting of AAV, as targeting integrins through RGD motifs [32] and the serpin receptor [56] has been demonstrated. Following characterization of six putative insertion sites, Girod et al. [32] inserted the L14 motif QAGTALRGDNPQG into position 1-587, recovered infectious particles and demonstrated increased infection of B16F10cells. In contrast, Wu et al. [56] inserted serpin receptor peptide ligands after amino acid 34 in VP1 and 138 in VP2 to produce genetically modified AAVs. However, although the inclusion of either peptide elevated transduction of the lung epithelial cell line IB3, both mutants required HSPG binding, suggesting that elevated transduction was due to use of the serpin receptor as a coreceptor [56]. This observation highlights two important points. First, there is an absolute requirement for use of peptide ligands that mediate primary receptor tethering of AAV to the cell surface by means of primary receptors independent of HSPG binding. Second, for peptides that can mediate cell binding but not internalization, modification of coreceptor usage to minimize degradation of internalized particles may be possible.

Here we found that SIGYPLP mediated enhanced, HSPG-independent transduction of human primary vascular EC from different venous beds. In the presence of heparin, AAVsig-mediated transduction was increased, which may indicate that a small amount of AAVsig infection is mediated by interaction with the less efficient wildtype MV pathway and that this infection can be rerouted through the SIGYPLP pathway in the presence of competing heparin. The lack of this potentiation effect at the higher dose of AAVsig may indicate a saturable route of transduction. Hence this peptide, originally isolated as an EC-binding moiety by phage display [47], maintained its targeting fidelity when inserted into the MV capsid. This is more remarkable when considering that the peptide was isolated from a linear phage display library. This directly demonstrates the ability of phage display to identify peptides that have the capacity to retarget gene therapy vectors to desired cell populations. In the case of SIGYPLP, it will be important for future development of the peptide to identify which amino acids are critical for EC binding to the receptor. A lack of knowledge of the identity of the receptor for SIGYPLP hampers efforts for further characterization of the method of transduction of SIGYPLP-modified MV vectors. Cloning of the receptor is of importance for both designing specific SIGYPLP pathway inhibitors to dissect the mechanism of transduction and identifying a receptor homologue in relevant animal models for gene transfer studies aimed at the endothelium in preclinical models.

Our finding of differential responses of AAVsig in comparison with AAVwt after inhibition of endosomal acidification (using bafilomycin A2) may indicate that intracellular trafficking of MV through the SIGYPLP pathway may be different from AAVwt. Bafilomycin A2 significantly elevated AAVsig-mediated gene transfer, but conversely reduced MVwt-mediated gene transfer supporting this theory. This hypothesis is further supported by the fact that SIGYPLP-modified plasmid/liposome complexes transduce HUVEC through a chloroquine-sensitive mechanism [68]. However, further in-depth investigations are required to dissect the intracellular trafficking of SlGYPLPmodified AAV.

We have demonstrated that a small peptide, originally isolated from a linear phage display library, can be incorporated into the capsid of MV vectors and maintain its targeting capacity. This study is the first to genetically modify MV vector tropism, using a single-component system, to an individual cell type pertinent to diverse human pathologies and has important implications for the design and use of AAV vectors in gene therapeutics targeted at the vasculature.

### Example 3

Previous studies have achieved Ad retargeting to vascular endothelium. Bispecific conjugates binding to candidate ligands such as E-selectin [11], basic fibroblast growth factor receptor [77] and integrins [6] have proven successful. A novel peptide, CGFECVRQCPERC, isolated by in vivo phage display in the mouse [28] and later demonstrated to bind endopeptidase [78] has also shown utility for retargeting via bispecific approaches [79]. We have previously reported the isolation of novel, human EC-binding peptides identified through phage display [47]. When fused to an antibody against the Ad5 fiber knob, the peptide SIGYPLP mediated adenoviral gene transfer into human EC at levels 15-fold higher than non-targeted Ad [47]. However, two-component systems such as these are subject to concerns of in vivo stability as well as the difficulty of producing consistent formulations.

Genetically modifying the Ad fiber would address these concerns, by producing retargeted vectors in a one-component system. Small targeting peptides can be readily inserted into the surface HI loop of the AdS fiber [34,9,76]. While these studies demonstrated addition of new vector tropism, in each case the resulting viruses were still able to bind CAR. A recent report identified a conserved receptor-binding site through motifs in the AB and CD loops on the fiber protein of CAR recognizing adenoviruses [80]. Further analysis identified that mutations in the DG loop of the knob abolished binding to CAR [81] and mutations in the AB, CD and FG loops also greatly reduced fiber-mediated gene transfer[74]. However, there are only two studies to date that combine CAR-binding ablation with insertion of a new targeting ligands [82,83]. Krasnykh et al [82] deleted the entire Ad5 fiber and replaced it with phage T4 fibritin. CAR binding was abolished, the trimerisation necessary for Ad particle formation was provided by a truncated version of phage T4 fibritin and retargeting was achieved via an artificial 6-His tag system [82]. Magnusson et al [83] deleted the knob and the final 15 C-terminal amino acids of the fiber shaft and utilised the neck region peptide of human lung surfactant protein D to provide trimerisation signals, linked to a RGD targeting motif.

Using an established transfection/infection system [74,75,84], we demonstrate the development of Ad vectors with novel mammalian EC-targeting peptides genetically engineered into the HI loop, in the absence or presence of additional CAR ablating point mutations. A stable re-targeted Ad vector with efficient and selective EC tropism was generated using the peptide SIGYPLP.

A. Materials and Methods

All chemicals were obtained from Sigma Chemical Co. (Poole, Dorset, UK) and cell culture reagents from Life Technologies (Paisley, Renfrewshire, UK) unless otherwise stated. Human umbilical vein endothelial cells (HUVEC), human saphenous vein endothelial cells (HSVEC), human vascular smooth muscle cells (VSMC), HeLa, 293T and HepG2 cell lines were cultured as described [47,90]. HAEC were obtained from TCS CellWorks (Botolph Claydon, Bucks. UK). Synthetic oligonucleotides were purchased from Operon (Alameda, Calif.).

DNA Constructs

Plasmid pDV111, containing the Ad5 fiber gene driven by the CMV promoter and a synthetic oligonucleotide encoding the 6 amino acid linker sequence GGSGGG inserted after amino acid T542 in the HI loop was used for insertion of targeting peptides. In detail, The Ad5 fiber open reading frame (ORF) was first cloned into pGEM3Zf(+) to create pGEM3Z/fiber, and site-directed mutagenesis [91] performed using 5′GGTACACAGGAAACAGGAGGTTCCGGAGGTGGAGGAGACACAACTCC3′ to create pDV18. This oligonucleotide includes a unique BspEI site (bold). The modified fiber gene was then amplified from pDV18 using oligonucleotide primers (fiber 5′ and fiber 3′) [90] and cloned into the BamHI and NotI sites of pCDNA3.1zeo(+). Finally, the Ad5 TPL was excised from pDV55 [75] and inserted at the BamHI site 5′ to the fiber gene to create pDV111. pDV115 and pV116 were created by cloning overlapping oligonucleotides encoding the sequences SIGYPLP (sense, 5′CCGGAAGCATCGGCTACCCCCTGCCCG3′ antisense, 5′CCGGCGGGCAGGGGGTAGCCGATGCTT3′) or LSNFHSS (sense, 5′CCGGACTGAGCMCTTCCACAGCTCCG3′; antisense 5′CCGGCGGAGCTGTGGMGTTGCTCAGT3′), respectively, into the BspEI site of pDV111. pDV110 is identical to pDV111 except that the fiber gene contains the KO2G point mutations (V441G and K442G), previously found to block CAR binding (Von Seggern, data not shown), which were introduced by overlap extension PCR using the primers KO2 top (5′GTTTCAGTTTTGGCCGGCGGGGGCAGTTTGGCTCC3′), KO2 bottom (5′GGAGCCAAACTGCCCCCGCCGGCCAAAACTGAAAC3′), fiber 5′ and fiber 3′ [90]. To construct pDV137, a fiber gene including both the HI linker residues and the KO1 point mutations (S408E and P409A), was generated by PCR overlap extension using the oligonucleotides fiber 5′, fiber 3′, KO1 top (5′GCTCCAGAGGCCMCTGCAGACTAAATG3′), and KO1 bottom (5′CATTTAGTCTGCAGTTGGCCTCTGGAGCTGG3′), with pDV111 as template. The HpaI-BspEI fragment of the new fiber gene, containing the KO1 mutations, was then used to replace the corresponding fragment of pDV111. The complementary synthetic oligonucleotides encoding the SIGYPLP peptide were annealed and inserted into the BspEI sites of pDV137 to create pDV138. All mutations and insertions were verified by sequence analysis.

Expression/Trimerization Analysis

We transfected fiber expressing plasmids into 293T cells using the GIBCO Calcium Phosphate System. 24 hours post transfection, cells were lysed in RIPA buffer [92] and soluble protein fractions electrophoresed under semi-native or denaturing conditions [90]. Gels were transferred to Immobilon-P membranes and probed with a polyclonal antibody against the Ad2 fiber (which cross reacts with the Ad5 fiber) as previously described [90].

Recombinant Adenovirus Production

We used a transient transfection/infection system [74] (FIG. 2A) to generate infectious particles of an eGFP-marked Ad vector containing fibers with HI loop insertions of the GGSGGG linker alone (AdCTL; pDV111-encoded), linker+SIGYPLP (AdSIG; pDV115-encoded), or linker+LSNFHSS (AdLSN; pDV116-encoded) fibers, as well as fibers defective in CAR binding (AdKO2G; pDV110-encoded) and AdKO1 (pDV137-encoded) and an Ad fiber simultaneously defective for CAR binding and SIGYPLP modified (AdKO1SIG; pDV138-encoded). For virus production, 293T cells were transfected with 20 μg/dish of the relevant plasmid using calcium phosphate and infected 24 hours later with 2000 particles/cell of the fiber-deleted vector Ad5.GFP.ΔF [84]. Virus was harvested by freeze/thaw lysis when the cytopathic effect was complete (−48 hours) and purified by ultracentrifugation through caesium chloride gradients. Virus particle number was determined using the established formula 1 μg protein=4×10⁹ viral particles.

Infections

We infected HUVEC, HSVEC, HAEC, VSMC, or HepG2 cells in 48 well plates (at ˜80% confluence) with increasing doses of Ad vectors for 3 or 16 hours. N.B. All multiplicities of infection are given in physical particles/cell. The media was changed and the cells lysed at the appropriate time point in 0.2% Triton-X-100. EGFP expression was quantified using a Wallac Victor2 with recombinant eGFP (Clontech, Basingstoke, UK) as a standard, or by western analysis using a rabbit polyclonal anti-eGFP antibody (Clontech).

Flow Cytometery

Cells were plated into 24 well plates and infected as described above. Cells were washed, trypsinised and resuspended in 0.5 ml PBS. Cell populations were analysed for eGFP fluorescence using a FACS scan.

Competitive Inhibition of CAR Binding

Cells were plated into 96 well plates 24 hours prior to infection. Cells were preincubated with 20 μg/ml soluble Ad5 fiber knob followed by infection with AdS as described above. At the required time point cells were lysed and reporter gene expression analysed by fluorimetry as described above.

Statistical Analysis

All data were analysed by unpaired Student's t test and are shown as mean±standard error of the mean (SEM). Data were considered significant when P<0.05. All experiments were performed in triplicate and repeated on at least 3 independent occasions.

B. Results

Analysis of Ad5 Transduction via CAR in EC and Non-EC Types

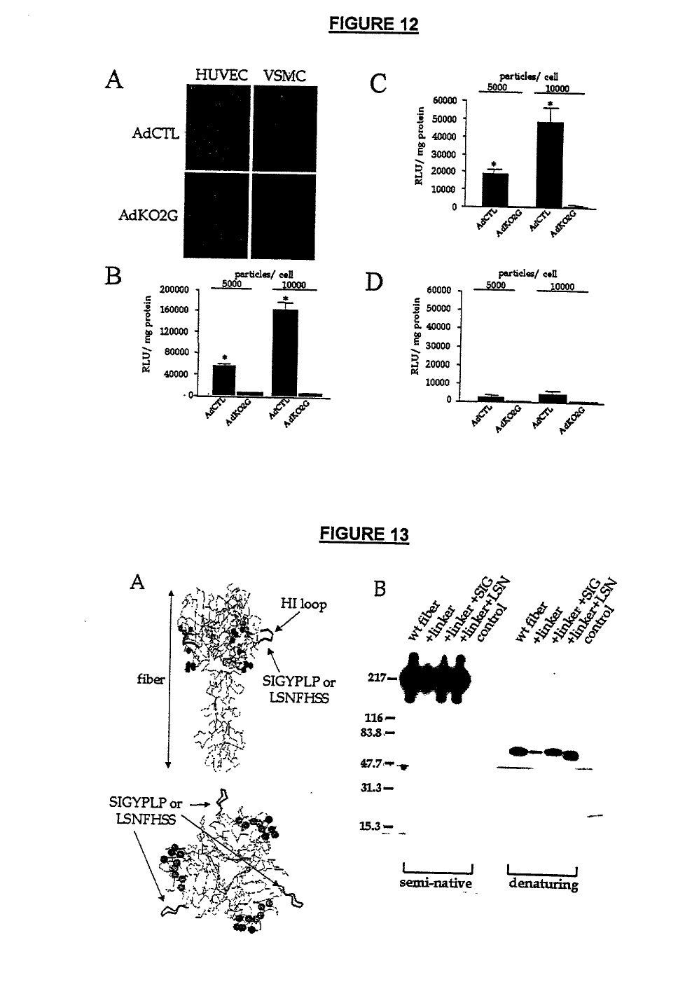

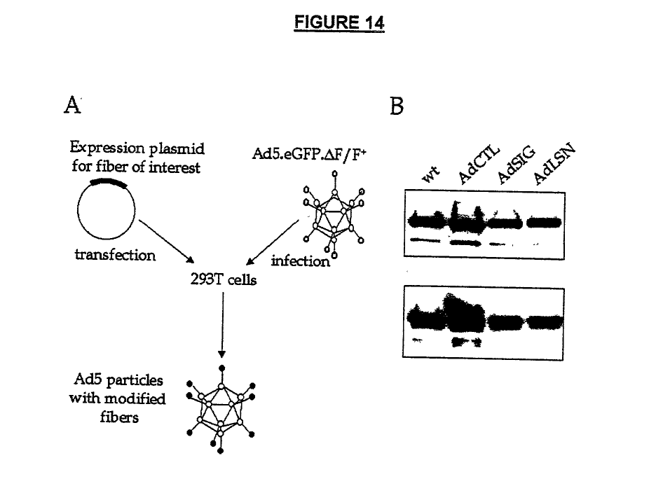

To assess the requirement for CAR binding in transduction of primary EC and non-EC types we transduced human umbilical vein EC (HUVEC), human saphenous vein EC (HSVEC) and human saphenous vein vascular smooth muscle cells (VSMC) either with the control adenovirus, AdCTL (which contains a wild type Ad5 fiber with the linker sequence ‘GGSGGG’ at T542 in the fiber's HI loop) or with a V441G and K442G mutated Ad, AdKO2G (Von Seggern, unpublished data) that blocks CAR binding (FIG. 12). Transduction with AdCTL was identical to an Ad vector with a non-modified fiber, indicating that linker insertion had no effect on transduction (data not shown). Using AdKO2G, transduction of both HUVEC and HSVEC demonstrated dependence on Ad-CAR binding (FIGS. 12A-C). In contrast, VSMC were poorly transduced by both AdCTL and AdKO2G, illustrating a lack of CAR-dependence (FIGS. 12A and D). We repeated the experiments with a second CAR-binding ablated Ad, AdKO1 (S408E and P409A [74]) with equivalent results (data not shown). We therefore reasoned that strategies to genetically modify Ad tropism selectively to EC required the use of efficient and highly selective EC-targeting peptides or ligands combined with the ablation of Ad-CAR binding.

Incorporation of EC-Targeting Peptides into the HI Loop

Previous work identified two EC-binding peptides (SIGYPLP and LSNFHSS) and demonstrated that SIGYPLP redirected Ad tropism to EC using a bispecific approach [47]. We inserted synthetic oligonucleotides encoding these sequences along with short, flexible linker after T542, which should result in their display within the exposed HI loop [34,9,76] (FIG. 13A). Fiber trimerization was first assessed (FIG. 13B). Under semi-native conditions antibody-reactive fiber trimers were detected (FIG. 13B). As expected, with denaturation ˜62 kDa monomers of all recombinant fibers were detected (FIG. 13B). We next used a versatile transient transfection/infection system (FIG. 14A) for production of Ads with genetically modified fibers [74,75]. 293T cells were transfected with DNA encoding the fiber of interest, then infected with an E1, E3 and L5 (fiber gene)-deleted Ad vector (Ad5.eGFP.ΔF) [84]. The resulting particles contain only that fiber produced by the transfected cells. Each fiber was efficiently incorporated into Ad particles, as evaluated by immunoblot analysis (FIG. 14B). Particles with the SIGYPLP- or LSNFHSS-modified fibers (AdSIG and AdLSN, respectively) had fiber levels comparable to non-modified fibers, or with a fibers containing the linker alone (AdCTL) (FIG. 14B).

AdSIG, but not AdLSN Demonstrates Enhanced EC Tropism

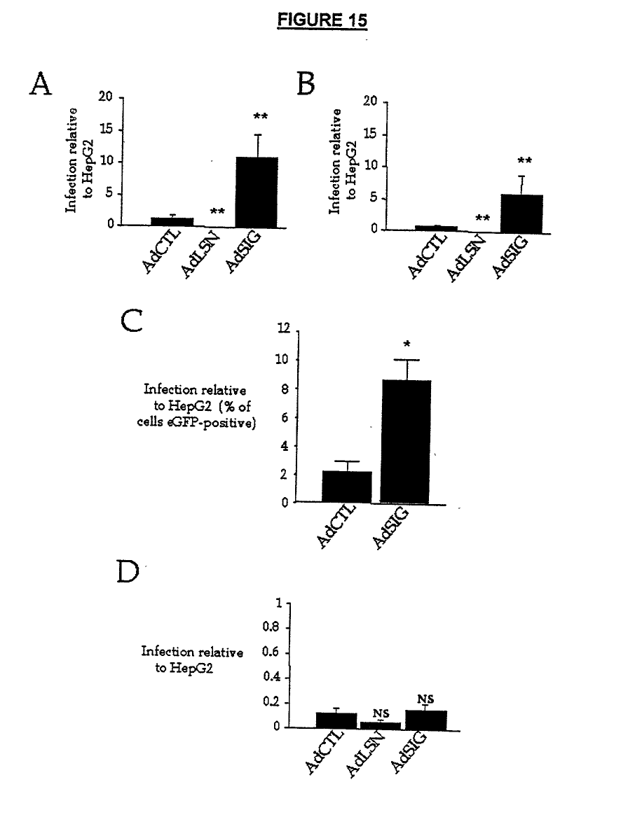

We compared reporter gene expression following infection with AdSIG and AdLSN to AdCTL (Table 3; FIG. 15).

TABLE 3
Reporter gene expression quantified from Ad-transduced cells.

Cell Type

| Virus | HUVEC | HSVEC | VSMC | HepG2 |
|---|---|---|---|---|
| AdCTL | 99.7 ± 26.6 | 68.7 ± 26.4 | 7.6 ± 3.3 | 135.3 ± 65.6 |
| AdSIG | 48.9 ± 17.2 | 23.7 ± 3.2 | 3.9 ± 2.0 | 4.5 ± 0.82 |
| AdLSN | 12.9 ± 4.0 | 4.4 ± 3.2 | 4.3 ± 1.5 | 623.3 ± 73.0 |

Levels of reporter gene expression in individual cell types after transduction with each Ad vector. Each cell type was transduced with 5000 particles/cell and reporter gene expression quantified 72 hours post infection. Results are presented as RLU (10³)/mg protein (mean ± S.E.M.) of experiments performed in triplicate on at least 3 independent occasions.

AdCTL produced high-level gene transfer in all cell types with the exception of VSMC (Table 3). However, AdSIG reporter gene expression in non-EC was severely reduced compared to AdCTL (Table 3). For example, reporter gene expression in HepG2 cells was 3.3% of that observed with AdCTL (Table 3). In HUVEC and HSVEC, however, reporter gene expression from AdSIG was 49% and 35%, respectively of that seen with AdCTL. These results suggested that insertion of the SIGYPLP peptide into the HI loop reduced CAR binding. The decrease in reporter gene expression in EC was much lower than that seen in HepG2 cells, suggesting the peptide was providing relative selectivity for EC. For AdLSN reporter gene expression was also decreased relative to AdCTL in all cell types with the exception of HepG2 cells (Table 3). Thus, LSNFHSS did not provide relative selectivity for EC. It is interesting to note that while the HI loop does not directly contact CAR, a small (2 amino acid) deletion in the Ad12 fiber's HI loop was able to reduce fiber-CAR interactions [85]. To analyse relative selectivity for EC we normalised the transduction in HUVEC and HSVEC to the transduction observed in HepG2 hepatocytes which express CAR, but do not support SIGYPLP or LSNFHSS-mediated gene transfer [47] (FIG. 15). AdSIG, but not AdLSN transduced HUVEC and HSVEC more efficiently relative to AdCTL (FIGS. 15A and B). This relative EC-selectivity evoked by AdSIG was confirmed by flow cytometry (FIG. 15C). To address the selectivity of the enhanced EC tropism, primary human VSMCs were also examined (FIG. 15D). In direct contrast to EC, the VSMC/HepG2 (FIG. 15D) infectivity ratio was not significantly altered from AdCTL (P=0.17). These experiments suggested that addition of the SIGYPLP peptide improved the selectivity of Ad transduction of EC compared to non-EC types. However, the AdSIG vector was still able to transduce HepG2 cells, highlighting the need to block CAR binding in order to achieve specific targeting as well as providing increased safety.

Simultaneous Detargeting and Retargeting of Ad Vectors

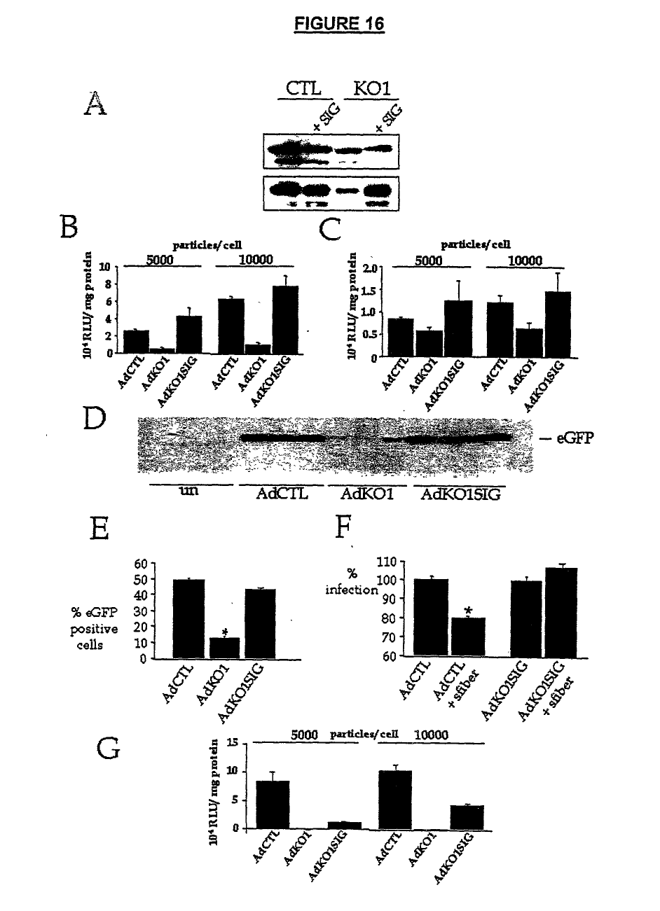

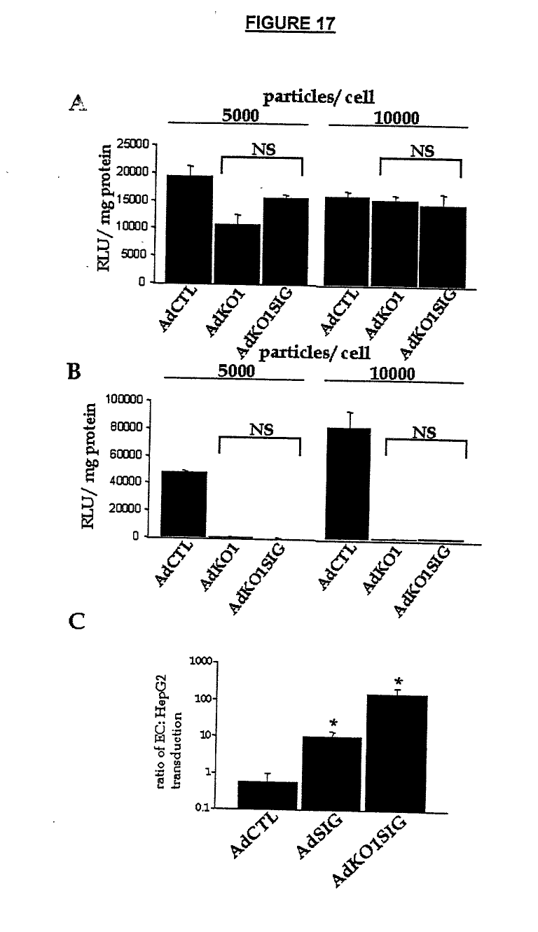

We attempted to produce EC-selective Ad fibers by combining point mutations that block CAR binding with insertion of the SIGYPLP peptide in the HI loop using the CAR-blocking mutations KO1 and KO2G, both of which reduce viral infectivity via CAR by ˜99% relative to non-modified Ad (KO1[74] and KO2G [Von Seggern, unpublished data]). HI loop insertion of SIGYPLP with the KO1 mutation produced Ad fibers that trimerised and assembled into mature virions (FIG. 16A). SIGYPLP peptide insertion with the KO2G mutation produced a fiber protein that failed to incorporate into viral particles (data not shown). We therefore used Ads with the KO1 mutation alone (AdKO1) or the additional SIGYPLP insertion (AdKO1SIG) to dissect gene transfer to EC (FIGS. 16B-E). AdKO1 had significantly reduced infectivity for EC (FIGS. 16B-E). Efficient transduction of EC was restored by AdKO1SIG (FIGS. 16B-E). AdKO1SIG produced a 4.7-fold (n=3, P<0.05) increase in gene transfer to HUVEC over AdKO1. Insertion of a second copy of SIGYPLP in tandem (AdKO1SIG₂) did not further enhance EC transduction (not shown). Western blotting and FACScan further confirmed the efficacy of SIGYPLP for EC transduction (FIGS. 16D and E). Competition experiments demonstrated soluble Ad5 fiber knob significantly inhibited AdCTL-, but not AdKO1ISIG-mediated EC reporter gene expression (FIG. 16F). We also quantified eGFP expression in Ad-transduced human aortic endothelial cells (HAEC) (FIG. 16G). In similarity to venous EC AdKO1SIG produced an 8-fold (n=3, P<0.05) increase in gene transfer in comparison to AdKO1, (FIG. 16G) demonstrating that SIGYPLP could mediate gene transfer to EC from diverse vascular beds. In VSMC and HepG2 cells AdKO1SIG-mediated reporter gene expression was not significantly different to AdKO1 (P=0.14 and P=0.35, respectively; FIGS. 17A and B), demonstrating the selective tropism of AdKO1SIG for EC. To determine whether blocking CAR binding in addition to peptide insertion further improved the selectivity of gene transfer for EC we normalised gene transfer of AdSIG or AdKO1SIG to the level observed for HepG2 cells (Table 4; FIG. 17C).

TABLE 4
Ratios of gene transfer from peptide-modified Ad vectors.

| Virus | Modification | HUVEC | HSVEC | HAEC | VSMC |
|---|---|---|---|---|---|
| AdCTL | — | 0.57 ± | 0.65 ± | 0.18 ± | 0.05 ± |
| | | 0.4 | 0.25 | 1.0 | 1.2 |
| AdSIG | HI insertion | 10.9 ± | 17.9 ± | N.D. | 0.16 ± |
| | | 3.6 | 6.0 | N.D. | 0.06 |
| AdKO1SIG | HI insertion + | 153.3 ± | 23.4 ± | 4.12 ± | 0.19 ± |
| | CAR ablation | 65.6 | 9.1 | 0.26 | 0.7 |

Ratio of gene transfer from each Ad vector in each cell type relative to that in HepG2 hepatocytes. Each cell type was infected with 5000 particles/cell and reporter gene expression was quantified 24 hours post-infection. Results are shown as mean ± S.E.M of experiments performed in triplicate on at least 3 independent occasions. ND. = not determined.

For HUVEC the ratio was improved from 10.9±3.6 for AdSIG, to 153.3±65.6 for AdKO1SIG, demonstrating a further improvement in selective-EC targeting of 14-fold. Comparing the ratios for AdKO1SIG versus AdCTL, the net improvement in EC-selectivity evoked by double genetic modification was 269-fold (Table 4; FIG. 17C). This selective shift in tropism was also confirmed by densitometric analysis of western blots (data not shown).

C. Discussion

For the full potential of gene therapy to be realised, major improvements to gene transfer vectors are a prerequisite. Development of vectors that can specifically target a cell or tissue of interest in vivo is especially important. The ability to genetically incorporate targeting peptides or motifs into the Ad capsid and to ablate its natural tropism would allow production of stable, versatile and cell-selective vectors with the potential for increased safety for in vivo gene delivery.

Analysis of the crystal structure of the Ad5 fiber knob [86] has revealed several exposed surface loops that are candidate sites for insertion of ligands. We, and others [34,76] have found that the exposed HI loop can accommodate peptide insertions to enable interaction with target cells. It is clear from our data that peptide modification within the HI loop of the Ad fiber may effect fiber-CAR binding. Based on the divergent effects we observed with SIGYPLP and LSNFHSS insertion, these effects are likely to be sequence dependent. Two recent studies have directly assessed the effect of HI loop modification on fiber-CAR interactions [80,85]. Roelvink et al [80] did not find any influence on CAR binding with a substituted HI loop in the Ad5 fiber knob. However, they deleted 12 amino acids (residues 538-549) and replaced them with just 4 (GSGG). Bewley et al [85] examined the Ad12 fiber/ CAR complex and observed that deletion of 2 amino acids within the HI loop made CAR binding weak, which they attributed to an indirect de-stabilisation of the CAR binding residues in the FG loop by the modified HI loop. The two EC-targeting peptides used in this study were originally isolated from a linear phage display library, [47], but are constrained at both their N- and C-termini when inserted into the HI loop, therefore peptide efficacy is likely determined by amino acid sequence and method of constraint. We found that combining point mutations to ablate CAR binding with SIGYPLP insertion resulted in a vast improvement in the selectivity of EC-transduction. Net improvement in the EC/HepG2 ratio of 269-fold for AdKO1 SIG versus AdCTL, compared to 19-fold for AdSIG versus AdCTL was observed. Hence, even with the use of efficient targeting ligands, additional mutations which ablate CAR binding are a prerequisite for production of safer Ad vectors.

Two previous studies have combined detargeting of Ad combined with retargeting in a single component vector [82,83], with both studies utilising large deletions within the fiber gene, not point mutations as described here. The first study, reported by Krasnykh et al [82], resulted in a vector that could mediate gene transfer to 293 cells expressing the anti-6-His tag scFv, however demonstrated reduced transduction of normal 293 cells in comparison to non-fiber modified Ad [82]. Magnusson et al [83] linked a RGD motif to a peptide that mediated fiber trimerisation and clearly demonstrated that the targeting moiety was exposed for binding and that levels of gene transfer into HeLa cells which express both CAR and □ᵥ□₅ integrins were approximately 70% of that observed with non-fiber modified Ad [83]. Furthermore, gene transfer into CAR negative, alphaᵥbeta₃ and alphaᵥbeta₅ integrin positive RD cells with the modified Ad was approximately 10-fold higher than that observed with non-fiber modified Ad [83]. It remains to be determined whether radical engineering of the Ad fiber is detrimental to essential steps in the Ad cell entry pathway such as endosomolysis.

In order to analyse the efficiency of SIGYPLP we quantified gene transfer in HUVEC, which were also the cell type from which SIGYPLP was originally isolated by phage display panning [47]. However, for in vivo gene delivery, the endothelium is a highly heterogeneous organ [28]. Therefore, to further examine the utility of SIGYPLP for EC-selective gene transfer we also analysed gene delivery into two other primary sources of human EC. We characterised gene transfer to another venous bed, human saphenous vein, implicated in vein bypass graft failure and we also examined gene transfer to HAEC. In both these EC-types SIGYPLP was able to mediate gene delivery, although the levels obtained differed, suggesting differential expression of the unidentified SIGYPLP receptor in the various EC. Thus targeting the receptor for SIGYPLP may be important for targeting EC's in vein graft disease as well as EC in the arterial system.

A recent report suggests that modification of Ad vectors to prevent CAR binding does not greatly change the pattern of tissue infection in vivo after systemic delivery [87]. This suggests that ablation of CAR-binding without addition of new tropism may not be enough to direct the virus away from the liver. However, addition of an RGD epitope to the HI loop did alter the distribution of systemically delivered Ad, even in the presence of a wild-type Ad fiber-CAR epitope [88].

It is clear from current literature that there is a lack of novel, mammalian cell selective ligands that retain their targeting efficacy when engineered into the Ad capsid. However, the peptide SIGYPLP does fulfil these criteria and furthermore also has utility for engineering into diverse gene transfer vectors, as we have also demonstrated that it retains its efficacy and selectivity when engineered into adeno-associated virus-2 (AAV-2) capsids [89].

In summary, this is the first study to combine Ad5 detargeting by point mutations, in combination with the genetic insertion of a mammalian cell-selective ligand, into an Ad5 vector in a single component system. This work has broad implications for the design and development of tropism-modified Ads to supply the demand for stable, genetically modified vectors for diverse gene-based therapeutics.

### Example 4

12 Amino Acid Peptides with Binding Specificity for Human Endothelial Cells

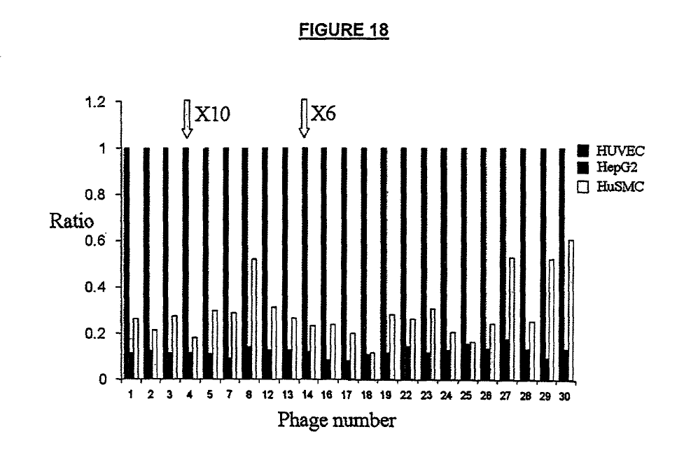

Biopanning was performed as previously detailed on human umbilical vein endothelial cells, using New England Biolabs 12 amino acid random phage display library. 5 rounds of panning were performed with pre-clearing step prior to first round. Individual phage were isolated, sequenced (see Table 5) and their affinity for endothelial cells v non-endothelial cells was ascertained (see FIG. 18).

TABLE 5

Peptide sequences obtained from panning human
umbilical vein endothelial cells (HEC).

| | | |
|---|---|---|
| No | SEQUENCE | REPEATS |

| | | |
|---|---|---|
| 1 | M A M P Q P A D H N N S | |
| 2 | V S G M S V P V Q L A R | |
| 3 | M T Q T P R T T P W P D | |
| 4 | M S L T T P P A V A R P | 6, 9, 11, 15, 33, |
| | | 34, 41, 47, 48 |
| | | (10 total) |
| 5 | M S N N P I R P P T S G | 10 |
| 6 | M S L T T P P A V A R P | |
| 7 | M T Q V Y T P P P T S T | |
| 8 | M T G S Q Q T L H P P P | |
| 9 | M S L T T P P A V A R P | |
| 10 | M S N N P I R P P T S G | |
| 11 | M S L T T P P A V A R P | |
| 12 | M A T Q P L S G S R L S (G) | |
| 13 | M N M T P P P H S P P K | |
| 14 | M T P F P T S N E A N L | 20, 31, 40, 51, 57 |
| | | (6 total) |
| 15 | M S L T T P P A V A R P | |
| 16 | A M S M T T M P H S P N | |
| 17 | M S D L L I E Y P P Y I | |
| 18 | M T L P H E L R D G A L | |
| 19 | A A V P P P Y V M S R P | |
| 20 | M T P F P T S N E A N L | |
| 21 | No peptide | |
| 22 | M S Q T P Y A R P Q Y V | |
| 23 | M T S N (P)H L N P G P R | |
| 24 | M G H N I N I P R T P L | |
| 25 | L S T P L P Y D M R R S | |
| 26 | M T R I Q D S P Y D L R | |
| 27 | M S T P P I R E Q A A H | |
| 28 | M T N L P T V T Q F P P | |
| 29 | M T P I A T S I P P Q M | |
| 30 | M T P T T P I P S L P Q | |
| 31 | M T P F P T S N E A N L | |
| 32 | M T S P H P Q T P N L T | |
| 33 | M S L T T P P A V A R P | |
| 34 | M S L T T P P A V A R P | |
| 35 | M T Q Q P P L P H P A K | |
| 36 | L A K P L P T T S N T G | |
| 37 | L S K P I P H I P S S I (G) | |
| 38 | C I C R G V G C C L L L | |
| 39 | L Q P P S M I T H P S T | 43 |
| 40 | M T P F P T S N E A N L | |
| 41 | M S L T T P P A V A R P | |
| 42 | L T P P N Q V L N P L Y | |
| 43 | L Q P P S M I T H P S T | |
| 44 | A F P M V G G P D H F R | |
| 45 | M L M P Q P A H H N N S | |
| 46 | A Q A M A N P L G S H I | |
| 47 | M S L T T P P A V A R P | |
| 48 | M S L T T P P A V A R P | |
| 49 | S S R I P G F P D P L H | |
| 50 | | |
| 51 | M T P F P T S N E A N L | |
| 52 | S M R G L P E L N P R I | |
| 53 | M S S P T V S S A P Q Y | |
| 54 | V L S M Q T P P T P L L | |
| 55 | T H A M S H L D K A H | |
| 56 | M A V Q P P N T S T S N | |
| 57 | M T P F P T S N E A N L | |
| 58 | M A I N D T Y P P P R P | |
| 59 | M M P P P T S L P S P S | |
| 60 | L A Q N P I Y R A H P H | |
| 61 | M Q P R P Q T L T P A S | |
| 62 | L T V P V P V S F A V H | |
| 63 | L T S P F S T P L N P R | 70 |
| 64 | No peptide | |
| 65 | M A G Q P K D S S K T L | |
| 66 | A N T P P H T I L S T E | |
| 67 | M G M T V P E N L I V Q | 68 |
| 68 | M G M T V P E N L I V Q | |
| 69 | M T P I Q S T Q Y P H S | |
| 70 | L T S P F S T P L N P R | |

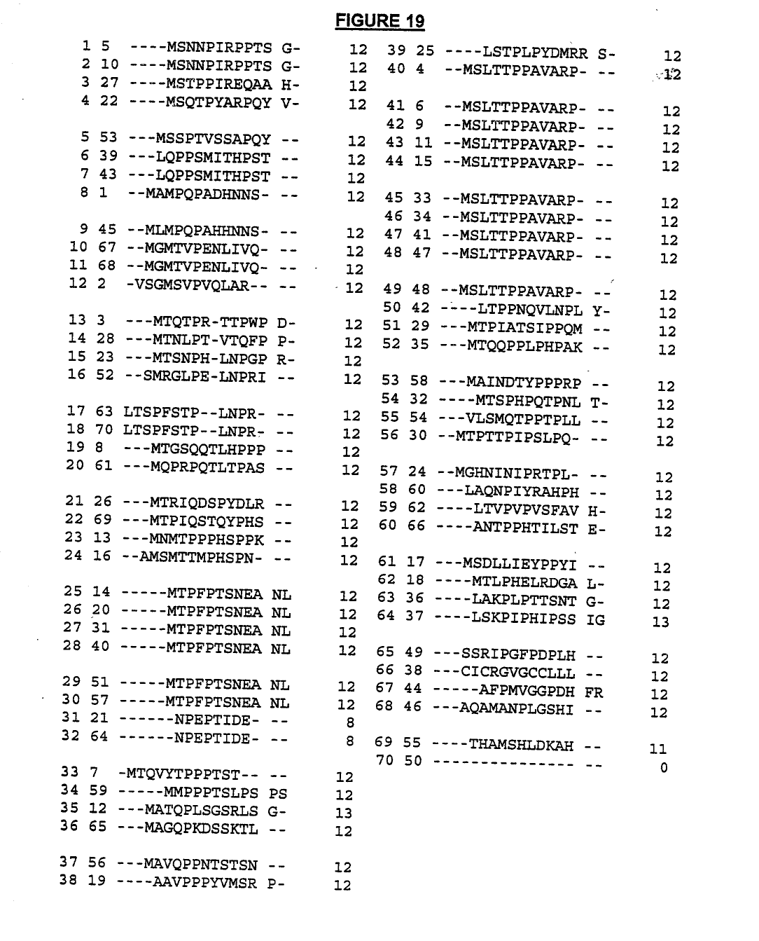

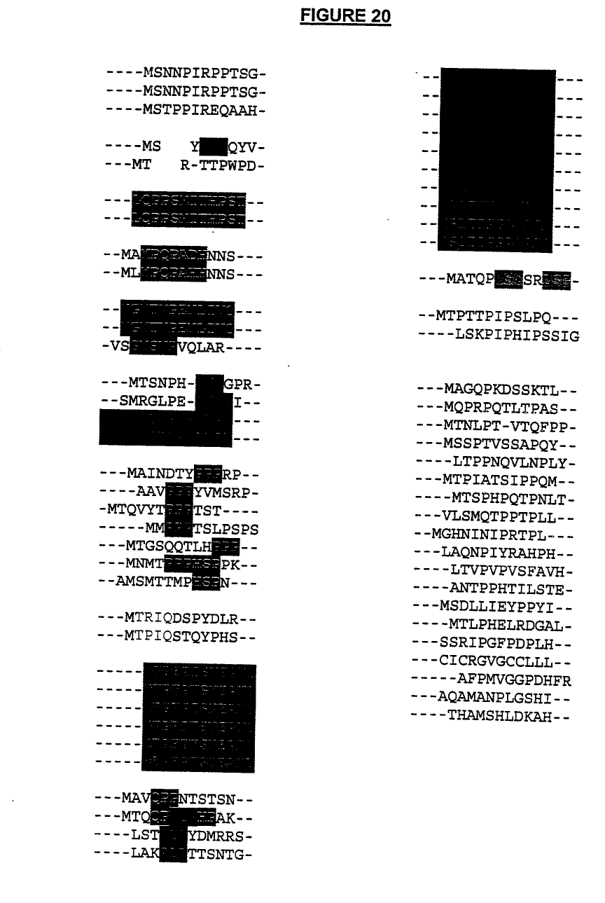

Seventy sequences were identified with 2 containing no peptides (21 and 64) and 1 rogue sequence (50). Of the remaining 67 peptide sequences, 18 were repeats with peptide 4 repeated 10 times, peptides 5, 39, 63 and 67 repeated twice, and peptide 14 repeated 6 times. This resulted in 49 different peptide ligands. The above sequences were aligned using the ClustalW multiple sequence alignment (courtesy of the BCM Search Launcher) (see FIG. 19) and the Fasta format (see FIG. 20). A number of motifs were identified throughout the peptides and which include PIR, QTP, ARP, MPQPAD/HH, GMS/TVP, LNP(R), PPP, HSP, LSG, PLP, TR/PIQ and QPP.

Although adenoviruses has been used as a model gene transfer system in this study, the isolated peptides disclosed herein may also be suitable for re-targeting gene transfer using non-viral vectors such as liposomes³¹ or other viral systems such as adeno-associated viruses³², retroviruses, lentiviruses and the like. In summary, novel peptides isolated from random phage display libraries can efficiently and effectively re-target gene transfer to cells normally relatively resistant to transduction, such as HEC. These data highlight the potential applications of small peptides in clinical gene therapy protocols where selective and efficient transduction of HEC would be advantageous.

References

1. Bergelson J M, Cunningham J A, Droguett G, Kurt-Jones E A, Krithivas A, Hong J S, Horwitz M S, Crowell R L, Finberg R W. Isolation of a common receptor for Coxsackie B viruses and adenoviruses 2 and 5. Science 1997;275:1320-1323.

2. Tomko R P, Xu R, Philipson L. HCAR and MCAR: The human and mouse cellular receptors for subgroup C adenoviruses and group B Coxsackieviruses. Proceedings of the National Academy of Sciences, USA. 1997;94:3352-3356.

3. Merrick A F, Shewring L D, Sawyer G J, Gustafsson K T, Fabre J W. Comparison of adenovirus gene transfer to vascular endothelial cells in cell culture, organ culture, and in vivo. Transplantation. 1996;62: 1085-1089.

4. Zinn K R, Douglas J T, Smyth C A, Liu H-G, Wu Q, Krasnykh V N, Mountz J D, Curiel D T, Mountz J M. Imaging and tissue biodistribution of ^{99m}Tc-labeled adenovirus knob (serotype 5). Gene Therapy. 1998;5:798-808.

5. Wickham T J, Roelvink P W, Brough D E, Kovesdi I. Adenovirus targeted to heparan-containing receptors increases its gene delivery efficiency to multiple cell types. Nature Biotechnology. 1996;14.

6. Wickham T J, Segal D M, Roelvink P W, Carrion M E, Lizonova A, Lee G M, Kovesdi I. Targeted adenovirus gene transfer to endothelial and smooth muscle cells by using bispecific antibodies. Journal of Virology. 1996;70:6831-6838.

7. Wickham T J, Lee G M, Titus J A, Sconocchia G, Bakacs T, Kovesdi I, Segal D M. Targeted adenovirus-mediated gene delivery to T cells via CD3. Journal of Virology. 1997;71 :7663-7669.

8. Wickham T J, Tzeng E, Shears II L L, Roelvink P W, Li Y, Lee G M, Brough D E, Lizonova A, Kovesdi I. Increased in vitro and in vivo gene transfer by adenovirus vectors containing chimeric fiber proteins. Journal of Virology. 1997;71:8221-8229.

9. Krasnykh V, Dmitriev I, Mikheeva G, Miller R, Belousova N, Curiel D T. Characterisation of an adenovirus vector containing a heterologous peptide epitope in the HI loop of the fiber knob. Journal of Virology. 1998;72:1844-1852.

10. Vigne E, Mahfouz I, Dedieu J-F, Brie A, Perricaudet M, Yeh P. RGD inclusion in the hexon monomer provides adenovirus type 5-based vectors with a fiber knob-independent pathway for infection. Journal of Virology. 1999;73:5156-5161.

11. Harari O A, Wickham T J, Stocker C J, Kovesdi I, Segal D M, Huehns T Y, Sarraf C, Haskard D O. Targeting an adenoviral gene vector to cytokine-activated vascular endothelium via E-selectin. Gene Therapy. 1999;6:801-807.

12. Rogers B E, Douglas J T, Sosnowski B A, Ying W, Pierce G, Buchsbaum D J, Della Manna D, Baird A, Curiel D T. Enhanced in vivo gene delivery to human ovarian cancer xenografts utilising a tropism-modified adenovirus vector. Tumor Targeting. 1998; 1:25-31.

13. Goldman C K, Rogers B E, Douglas J T, Sosnowski B A, Ying W, Siegal G P, Baird A, Campain J A, Curiel D T. Targeted gene delivery to Kaposi's sarcoma cells via the fibroblast growth factor receptor. Cancer Research. 1997;57:1447-1451.

14. Jaffe E A. Culture of human endothelial cells derived from umbilical vein: identification by morphologic and immunologic criteria. Journal of Clinical Investigation. 1973;52:2745-2756.

15. Southgate K, Newby A C. Serum-induced proliferation of rabbit aortic smooth muscle cells from the contractile state is inhibited by 8-Br-cAMP but not 8-Br-cGMP. 1990;82:113-123.

16. Watkins S J, Mesyanzhinov V, Kurochkina L P, Hawkins R E. The adenobody approach to viral targeting: specific and enhanced adenoviral delivery. Gene Therapy. 1997;4: 1004-1012.

17. Wilkinson G W G, Akrigg A. Constitutive and enhanced expression from the CMV major IE promoter in a defective adenovirus vector. Nucleic Acids Research. 1992;20:2233-2239.

18. Cwirla S E, Peters E A, Barrett R W, Dower W J. Peptides on phage: a vast library of peptides for identifying ligands. Proceedings of the National Academy of Sciences, USA. 1990;87:6378-6382.

19. Koivunen E, Wang B, Ruoslahti E. Isolation of a highly specific ligand for the alpha₅beta₁ integrin from a phage display library. The Journal of Cell Biology. 1994;124:373-380.

20. Barry M A, Dower W J, Johnston S A. Toward cell-targeting gene therapy vectors: selection of cell-binding peptides from random peptide-presenting phage libraries. Nature Medicine. 1996;2:299-305.

21. Palmer D B, George A J T, Ritter M A. Selection of antibodies to cell surface determinants on mouse thymic epithelial cells using a phage display library. Immunology. 1997;91:473-478.

22. Szardenings M, Tornroth S, Mutulis F, Muceniece R, Keinanen K, Kuusinen A, Wikberg J E S. Phage display on whole cells yields a peptide specific for melanocortin receptor 1. The Journal of Biological Chemistry. 1997:27943-27948.

23. Douglas J T, Rogers B E, Rosenfeld M E, Michael Si, Feng M Z, Curiel D T. Targeted gene delivery by tropism-modified adenoviral vectors. Nature Biotechnology. 1996;14:1574-1578.

24. Cheresh D, Spiro R. Biosynthetic and functional properties of an Arg-Gly-Asp-directed receptor involved in human-melanoma cell attachment to vitronectin, fibrinogen and von Willebrand factor. Journal of Biological Chemistry. 1987;262:17703-17711.

25. Gladson C L, Cheresh D A. The alphaᵥ integrins. In: Y. Takeda, ed. Integrins: the biological problems. Boca Raton, Fla.: CRC Press; 1994:83-99.

26. Pasqualini R, Ruoslahti E. Organ targeting in vivo using phage display peptide libraries. Nature. 1996;380:364-367.

27. Pasqualini R, Koivunen E, Ruoslahti E. Alpha v integrins as receptors for tumor targeting by circulating ligands. Nature Biotechnology. 1997;15:542-546.

28. Rajotte D, Arap W, Hagedorn M, Koivunen E, Pasqualini R, Ruoslahti E. Molecular heterogeneity of the vascular endothelium revealed by in vivo phage display. Journal of Clinical Investigation. 1998;102:430-437.

29. Arap W, Pasqualini R, Ruoslahti E. Cancer treatment by targeted drug delivery to tumor vasculature in a mouse model. Science. 1998;279:377-380.

30. Wickham T J, Carrion M E, Kovesdi I. Targeting of adenovirus penton base to new receptors through replacement of its RGD motif with other receptor-specific peptide motifs. Gene Therapy. 1995;2:750-756.

31. Hart S, Arancibia-Carcamo C, Wolfert M, Mailhos C, Oreilly N, Ali R, Coutelle C, George A, Harbottle R, Knight A, Larkin D, Levinsky R, Seymour L, Thrasher A, Kinnon C. Lipid-mediated enhancement of transfection by a nonviral integrin-targeting vector. Human Gene Therapy. 1998;9:575-585.

32. Girod A, Ried M, Wobus C, Lahm H, Leike K, Kleinschmidt J, Deleage G, Hallek M. Genetic capsid modifications allow efficient re-targeting of adeno-associated virus type 2. Nature Medicine. 1999;5:1052-1056.

33. Romanczuk H, Galer C E, Zabner J, Barsomian G, Wadsworth S C and O'Riordan, C R. Modification of an adenoviral vector with biologically selected peptides: a novel strategy for gene delivery to cells of choice. Human Gene Therapy. 1999; 10: 2615-2626.

34. Dmitriev I, Krasnykh V, Miller C R, Wang M, Kashentseva E, Mikheeva G, Belousova N and Curiel D T. An adenovirus vector with genetically modified fibers demonstrates expanded tropism via utilisation of a Coxsackie and adenovirus receptor-independent cell entry mechanism. Journal of Virology. 1998; 72: 9706-9713.

35. Bartlett J S, Kleinschmidt J, Boucher R C and Samulski R J. Targeted adeno-associated virus vector transduction of non-permissive cells mediated by a specific F(ab'γ)(2) antibody. Nature Biotechnology. 1999; 17: 181-186.

36. Hall F L, Gordon E M, Wu L, Zhu N L, Skotzko M J, Starnes V A, and Anderson W F. Targeting retroviral vectors to vascular lesions by genetic engineering of the MoMLV gp70 envelope protein. Human Gene Therapy. 1997; 8: 2183-2192.

37. Kasahara N, Dozy A M, Wai Kan Y. Tissue-specific targeting of retroviral vectors through ligand-receptor interactions. Science. 1994; 266: 375-378.

38. Valsesia-Wittmann S, Drynda A, Deleage G, Aumailley M, Heard J M, Danos O, Verdier G and Cosset F L. Modifications in the binding domain of avian retrovirus envelope protein to redirect the host range of retroviral vectors. Journal of Virology. 1994; 68: 4609-4619.

39. Hart S L, Collins L, Gustafsson K, Fabre J W. Integrin-mediated transfection with peptides containing arginine-glycine-aspartic acid domains. Gene Therapy. 1997; 4: 1225-1230.

40. Crompton J, Toogood C I A, Wallis N and Hay R T. Expression of a foreign epitope on the surface of the adenovirus hexon. Journal of General Virology. 1994; 75, 133-139.

41. Eavarone D A, Yu X J, Bellamkonda R V. Targeted drug delivery to C6 glioma by transferrin-coupled liposomes. Journal of Biomedical Materials Research. 2000; 51: 10-14.

42. Tsunoda S, Ohizumi I, Matsui J, Koizumi K, Wakai Y, Makimoto H, Tsutsumi Y, Utoguchi N, Tamiguchi K, Sato H, Hanada N, Ohsugi Y and Magumi T. Specific binding of TES-23 antibody to tumor vascular endothelium in mice, rats and human cancer tissue. A novel drug carrier for cancer targeting therapy. British Journal of Cancer. 1999; 81: 1155-1161.

43. Kaplitt, M., et al. (1994). Long-term gene expression and phenotypic correction using adeno-associated virus vectors in the mammalian brain. Nat Genet. 8: 148-154.

44. Kessler, P. D., et al. (1996). Gene delivery to skeletal muscle results in sustained expression and systemic delivery of a therapeutic protein. Proc. Natl. Acad. Sci. USA. 93: 14082-14087.

45. Snyder, R. O., et al. (1999). Correction of hemophilia B in canine and murine models using recombinant adeno-associated viral vectors. Nat. Med. 5: 64-70.

46. Herzog, R., et al. (1999). Long-term correction of canine hemophila B by gene transfer of blood coagulation factor IX mediated by adeno-associated viral vector. Nat. Med. 5: 56-63.

47. Nicklin, S. A., White, S. J., Watkins, S. J., Hawkins, R. E., and Baker, A. H. (2000). Selective targeting of gene transfer to vascular endothelial cells by use of peptides isolated by phage display. Circulation 102: 231-237.

48. Wen, S., et al. (2000). Second-generation adenoviral vectors do not prevent rapid loss of transgene expression and vector DNA from the arterial wall. Arterioscler. Thromb. Vasc. Biol. 20: 1452-1458.

49. Tang, X., et al. (1999). Intravenous angiotensinogen antisense in AAV-based vectors decreases hypertension. Am. J. Physiol. 277: 2392-2399.

50. Mohuczy, D., Gelband, C. H., and Phillips, I. M. (1999). Antisense inhibition of AT1 receptor in vascular smooth muscle cells using adeno-associated virus-based vector. Hypertension 33: 354-359.

51. Richter, M., et al. (2000). Adeno-associated virus vector transduction of vascular smooth muscle cells in vivo. Physiol. Genomics 2: 117-127.

52. Maeda, Y., et al. (1997). Gene transfer into vascular cells using adeno-associated virus (AAV) vectors. Cardiovasc. Res. 35: 514-521.

53. Summerford, C., and Samulski, R. J. (1998). Membrane-associated heparan sulfate proteoglycan is a receptor for adeno-associated virus type 2 virions. J. Virol. 72: 1438-1445.

54. Summerford, C., Bartlett, J. S., and Samulski, R. J. (1999). alphaᵥbeta₅ integrin: a co-receptor for adeno-associated virus type 2 infection. Nat. Med. 5: 78-82.

55. Qing, K., et al. (1999). Human fibroblast growth factor receptor-1 is a co-receptor for infection by adeno-associated virus 2. Nat Med. 5: 71-77.

56. Wu, P., et al. (2000). Mutational analysis of the adeno-associated virus type 2 (AAV2) capsid gene and construction of AAV2 vectors with altered tropism. J. Virol. 74: 8635-8647.

57. Lynch, C. M., et al. (1997). Adeno-associated virus vectors for vascular gene delivery. Circ. Res. 80: 497-505.

58. Nakai, H., et al. (1998). Adeno-associated viral vector-mediated gene transfer of human blood coagulation factor IX into mouse liver. Blood 91: 4600-4607.

59. Nathwani, A., Davidoff, A., Hanawa, H., Zhou, J.-F., and Nienhuis, A. W. (2001). Factors influencing in vivo transduction by recombinant adeno-associated vectors expressing the human factor IX cDNA. Blood 97: 1258-1265.

60. Bartlett, J. S., Wilcher, R., and Samulski, R. J. (2000). Infectious entry pathway of adenoassociated virus and adeno-associated virus vectors. J. Virol. 74: 2777-2785.

61. Duan, D., Yue, Y., Yan, Z., and Engelhardt, J. F. (2000). Endosomal processing limits gene transfer to polarised airway epithlelia by adeno-associated virus. J. Clin. Invest. 105: 1573-1587.

62. Douar, A.-M., Poulard, K., Stockholm, D., and Danos, O. (2001). Intracellular trafficking of adeno-associated virus vectors: routing to the late endosomal compartment and proteasome degradation. J. Virol. 75: 1824-1833.

63. Wobus, C. E., et al. (2000). Monoclonal antibodies against the adeno-associated virus type 2 (AAV-2) capsid: epitope mapping and identification of capsid domains involved in AAV-2-cell interaction and neutralization of AAV-2 infection. J. Virol. 74: 9281-9293.

64. Moskalenko, M., et al. (2000). Epitope mapping of human anti-adeno-associated virus type 2 neutralizing antibodies: implications for gene therapy and virus structure. J. Virol. 74: 1761-1766.

65. Yang, Q. C., et al. (1998). Development of novel cell surface CD34-targeted recombinant adeno-associated virus vectors for gene therapy. Hum. Gene Ther. 9: 1929-1937.

66. Bartlett, J. S., Kleinschmidt, J., Boucher, R. C., and Samulski, R. J. (1999). Targeted adenoassociated virus vector transduction of non-permissive cells mediated by a bi-specific F(ab'γ)(2) antibody. Nat. Biotechnol. 17: 181-186.

67. Reynolds, P., et al. (2000). A targetable, injectable adenoviral vector for selective gene delivery to pulmonary endothelium in vivo. Mol. Ther. 2: 562-578, doi:10.1006/mthe.2000.0205.

68. Parker, A., et al. (2001). Enhanced gene transfer activity of poly(L-lysine)/DNA complexes targeted using an oligopeptide identified by phage panning. J. Gene Med. 2: 67.

69. Samulski, R. J., Chang, L., and Shenk, T. (1987). A recombinant plasmid from which an infectious adeno-associated virus genome can be excised in vitro and its use to study viral replication. J. Virol. 61: 3096-3101.

70. Chiorini, J., et al. (1995). High-efficiency transfer of the T cell costimulatory molecule B7-2 to lymphoid cells using high-titer recombinant adeno-associated vectors. Hum. Gene Ther. 6: 1531-1541.

71. Xiao, X., Li, J., and Samulski, R. J. (1998). Production of high-titer recombinant adenoassociated virus vectors in the absence of helper adenovirus. J. Virol. 72: 2224-2232.

72. Ali, R., et al. (2000). Restoration of photoreceptor ultrastrucure and function in retinal degeneration slow mice by gene therapy. Nat. Genet. 25: 306-310.

73. Zolotukhin, S., et al. (1999). Recombinant adeno-associated virus purification using novel methods improves infectious titer and yield. Gene Ther. 6: 973-985.

74. Jakubczak, J. L., et al. (2001). Adenovirus type 5 particles pseudotyped with mutagenised fiber proteins show diminished infectivity of coxsackie B-adenovirus receptor-bearing cells. Virology. 75:2972-2981.

75. Von Seggern, D. J., Huang, S., Kaye-Fleck, S., Stevenson, S. C., and Nemerow, G. R. (2000). Adenovirus vector pseudotyping in fiber expressing cell lines: improved transduction of Epstein-Barr virus-transformed B cells. J. Virol. 74:354-362.

76. Xia, H., Anderson, B., Mao, Q., and Davidson, B. L. (2000). Recombinant human adenovirus: targeting to the human transferrin receptor improves gene transfer to brain microcapillary endothelium. J. Virol. 74:11359-11366.

77. Reynolds, P. N., et al. (1998). Targeting adenoviral infection with basic fibroblast growth factor enhances gene delivery to vascular endothelial and smooth muscle cells. Tumor Targeting. 3:156-168.

78. Rajotte, D., and Ruoslahti, E. (1999). Membrane dipeptidase is the receptor for a lung-targeting peptide identified by in vivo phage display. J. Biol. Chem. 274:11593-11598.

79. Trepel, M., Grifman, M., Weitzman, M. D., and Pasqualini, R. (2000). Molecular adaptors for vascular-targeted adenoviral gene delivery. Hum. Gene Ther. 11:1971-1981.

80. Roelvink, P. W., Mi, L. G., Einfeld, D. A., and Wickham, T. J. (1999). Identification of a conserved receptor-binding site on the fiber protein of CAR-recognizing adenoviridae. Science. 286:1568-1571.

81. Kirby, I., et al. (1999). Mutations in the DG loop of adenovirus type 5 fiber knob protein abolish high-affinity binding to its cellular receptor CAR. J. Virol. 73:9508-9514.

82. Krasnykh, V., Belousova, N., Korokhov, N., Mikheeva, G., and Curiel, D. T. (2001). Genetic targeting of an adenovirus vector via replacement of the fiber protein with phage T4 fibritin. J. Virol. 75:4176-4183.

83. Magnusson, M. K., Hong, S. S., Boulanger, P., and Lindholm, L. (2001). Genetic retargeting of adenovirus: novel strategy employing “deknobbing” of the fiber. J. Virol. 75:7280-7289.

84. Wu, E., Fernandez, J., Kaye Fleck, S., Von Seggern, D. J., Huang, S., and Nemerow, G. R. (2001). A 50-kDa membrane protein mediates sialic acid-independent binding and infection of conjunctival cells by adenovirus type 37. Virology. 279:78-89.

85. Bewley, M. C., Springer, K., Zhang, Y.-B., Freimuth, P. and Flanagan, J. M. (1999). Structural analysis of the mechanism of adenovirus binding to its human cellular receptor, CAR. Science. 286:1579-1583.

86. Xia, D., Henry, L. J., Gerard, R. D., and Deisenhofer, J. (1994). Crystal structure of the receptor-binding domain of adenovirus type 5 fiber protein at 1.7 Å resolution. Structure. 2: 1259-1270.

87. Leissner, P., et al. (2001). Influence of adenoviral fiber mutations on viral encapsidation, infectivity and in vivo tropism. Gene Ther. 8:49-57.

88. Reynolds, P. N., Dmitriev, I., and Curiel, D. T. (1999). Insertion of an RGD motif into the HI loop of adenovirus fiber protein alters the distribution of transgene expression of the systemically administered vector. Gene Ther. 6:1336-1339.

89. Nicklin, S. A., et al. (2001). Efficient and selective AAV2-mediated gene transfer directed to human vascular endothelial cells. Mol. Ther. 4:174-181. doi:10.1006/mthe.2001.0424.

90. Von Seggern, D. J., Kehler, J., Endo, R. L., and Nemerow, G. R. (1998). Complementation of a fibre mutant adenovirus by packaging cell lines expressing the adenovirus type 5 fibre protein. J. Gen. Virol. 79:1461-1468.

91. Kunkel, T. (1985). Rapid and efficient site-specific mutagenesis without phenotypic selection. Proc. Nat. Acad. Sci. USA 82:488-492.

92. Harlow, E., and Lane, D. (1988). Antibodies. Cold Spring Harbour Laboratory, Cold Spring Harbor, N.Y.

1. A targeting peptide comprising an amino acid sequence selected from the group consisting of:
AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or a derivative thereof, for targeting a material to a cell.

2. A targeting peptide according to claim 1 wherein said peptide comprises an amino acid sequence selected from the group consisting of SIGYPLP, NTGPNTV, LSNFHSS, GILSPSH MSSPGPA, MSLTTPPAVARP, MTPFPTSNEANL and MGMTVPENLIVQ, or a derivate thereof.

3. A targeting peptide according to claim 1 wherein said peptide comprises an amino acid sequence selected from the group consisting of SIGYPLP, NTGPNTV, LSNFHSS, GILSPSH and MSSPGPA, or a derivate thereof.

4. A targeting peptide according to claim 1 wherein said peptide comprises an amino acid sequence selected from the group consisting of MSLTTPPAVARP, MTPFPTSNEANL and MGMTVPENLIVQ, or a derivate thereof.

5. A targeting peptide according to claim 1 wherein said peptide is up to 100 amino acids long.

6. A targeting peptide according to claim 1 wherein said cell is a vascular endothelial cell.

7. A targeting peptide according to claim 1 wherein said material to be targeted to a cell is selected from the group consisting of drug delivery vehicles, gene therapy vehicles, bacteria, non-ionic surfactant vesicles, microcapsules and vaccination products.

8. A pharmaceutical composition comprising a targeting peptide in association with a vehicle, the targeting peptide comprising an amino acid sequence selected from the group consisting of:
AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or a derivative thereof, the vehicle carrying a pharmaceutically active agent, and a pharmaceutically acceptable carrier.

9. A pharmaceutical composition according to claim 8 wherein said peptide comprises an amino acid sequence selected from the group consisting of SIGYPLP, NTGPNTV, LSNFHSS, GILSPSH MSSPGPA, MSLTTPPAVARP, MTPFPTSNEANL and MGMTVPENLIVQ, or a derivate thereof.

10. A pharmaceutical composition according to claim 8 wherein said peptide comprises an amino acid sequence selected from the group consisting of SIGYPLP, NTGPNTV, LSNFHSS, GILSPSH and MSSPGPA, or a derivate thereof.

11. A pharmaceutical composition according to claim 8 wherein said peptide comprises an amino acid sequence selected from the group consisting of MSLTTPPAVARP, MTPFPTSNEANL and MGMTVPENLIVQ, or a derivate thereof.

12. A pharmaceutical composition according to claim 8 wherein said peptide is up to 100 amino acids long.

13. A pharmaceutical composition according to claim 8 wherein said pharmaceutically active agent is selected from the group consisting of a biologically active drug, a further peptide(s) and polynucleic acid.

14. A pharmaceutical composition according to claim 8 wherein the targeting peptide is in direct association with the vehicle carrying a pharmaceutically active agent.

15. A pharmaceutical composition according to claim 8 wherein the targeting peptide is indirectly associated with the vehicle carrying the pharmaceutically active agent.

16. A pharmaceutical composition according to claim 8 wherein the targeting peptide is covalently bound to the pharmaceutically active agent.

17. A pharmaceutical composition according to claim 8 wherein said composition is used to treat mammals.

18. A pharmaceutical composition according to claim 17 wherein said composition is used to treat humans.

19. A method of targeting a material to cell, said method comprising bringing into association a targeting peptide comprising amino acid sequence selected from the group consisting of:
AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or a derivative thereof, with the material to be targeted to form a complex and exposing the complex to a cell(s).

20. A method according to claim 19 wherein said targeting peptide comprises an amino acid sequence selected from the group consisting of SIGYPLP, NTGPNTV, LSNFHSS, GILSPSH MSSPGPA, MSLTTPPAVARP, MTPFPTSNEANL and MGMTVPENLIVQ, or a derivate thereof.

21. A method according to claim 19 wherein said targeting peptide comprises an amino acid sequence selected from the group consisting of SIGYPLP, NTGPNRV, LSNFHSS, GILSPSH and MSPPGPA or a derivative thereof.

22. A method according to claim 19 wherein said targeting peptide comprises an amino acid sequence selected from the group consisting of MSLTTPPAVARP, MTPFPTSNEANL and MGMTVPENLIVQ, or a derivative thereof.

23. A method according to claim 19 wherein said targeting peptide is up to 100 amino acids long.

24. A method according to claim 19 wherein the cell to be targeted is a endothelial cell.

25. A method according to claim 19 wherein said endothelial cell is a vascular endothelial cell.

26. A method according to claim 19 wherein said method is performed in vivo.

27. A method according to claim 26 wherein said method is used to target a material selected from the group consisting of drug delivery vehicles, gene therapy vehicles, bacteria, non-ionic surfactant vesicles, microcapsules and vaccine products.

28. A kit comprising a targeting peptide comprising an amino acid sequence selected from the group consisting of:
AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or a derivative thereof, for transfecting or identifying cell types in vitro.

29. A kit according to claim 28 wherein said targeting peptide comprises an amino acid sequence selected from the group consisting of SIGYPLP, NTGPNTV, LSNFHSS, GILSPSH MSSPGPA, MSLTTPPAVARP, MTPFPTSNEANL and MGMTVPENLIVQ, or a derivate thereof.

30. A kit according to claim 28 wherein said targeting peptide comprises an amino acid sequence selected from the group consisting of SIGYPLP, NTGPNRV, LSNFHSS, GILSPSH and MSPPGPA or a derivative thereof.

31. A kit according to claim 28 wherein said targeting peptide comprises an amino acid sequence selected from the group consisting of MSLTTPPAVARP, MTPFPTSNEANL and MGMTVPENLIVQ, or a derivative thereof.

32. A kit according to claim 28 wherein said targeting peptide is up to 100 amino acids long.

33. A kit according to claim 28 wherein said targeting peptide further comprises a linking region for binding molecular groups.

34. A kit according to claim 33 wherein said molecular groups are selected from the group consisting of reagents, pharmaceutically active agents, vesicles, diagnostic markers and antibodies.

35. A method for screening targeting peptides capable of binding to an endothelial cell, said method comprising:
inserting a polynucleotide encoding a potential endothelial cell-binding peptide into an expression vector;
expressing the peptide;
conducting a pre-screening step with the expressed peptides using non-endothelial cells in order to select for the expressed peptides with reduced or negligible binding to the non-endothelial cells;
further screening the expressed peptides which exhibited reduced or negligible binding to the non-endothelial cells using endothelial cells; and
selecting for the expressed peptides which display selective and efficient binding to the endothelial cells.

36. A method according to claim 35 wherein said endothelial cells are human endothelial cells.

37. A method according to claim 35 wherein said non-endothelial cells are selected from a group consisting on human vascular smooth muscle cells and hepatocytes.

38. A gene therapy vector, said vector comprising a targeting peptide as described in claim 1, a vehicle associated with the targeting peptide and a nucleotide sequence comprising the gene for targeting carried within said vehicle.

39. A method of treating a disease, said method comprising administering a pharmaceutical agent in association with a targeting peptide, said targeting peptide comprising amino acid sequence selected from the group consisting of:
AASARLP, VYFPAPN, FSMSTPS, IVAQPRL, FPQTYTT, NIAAFSL, QPRLLHH, NIIPAPT, SPTYPRR, TRSQPPL, NTGPNRV, PPPDWTF, SHFSHLR, AFNYPPH, DFLQVSP, SPDHLFC, LEHPPTT, TYPSSEW, IPMHLHN, TSESPTV, YSLSRSL, NHLSALY, TYSLKSA, TSTMPSR, ETIKTNT, ATGFATP, TNSQPSP, TSFFMPP, TAAYRFW, LPPSLYS, SPSVVPF, HSLTFSI, WNSTTQA, HFTHPTH, AGATAMS, STYPIIR, SWNHARV, NHWHGGL, GILSPSH, EAVPTYS, INSNAPG, YSTHSTR, SDLATVR, INSVSPH, MSSPGPA, LPTKTLF, AAWPTSS, LTAELTP, KIDGTPR, VEPARAS, SIGYPLP, WTSDELH, TLGLHMS, LSNFHSS, SLPRNSD, GYQQVFQ, MSPPGPA, LCMTTLV, SEVAVQG, MAMPQPADHNNS, VSGMSVPVQLAR, MTQTPRTTPWPD, MSLTTPPAVARP, MSNNPIRPPTSG, MTQVYTPPPTST, MTGSQQTLHPPP, MATQPLSGSRLSG, MNMTPPPHSPPK, MTPFPTSNEANL, AMSMTTMPHSPN, MSDLLIEYPPYI, MTLPHELRDGAL, AAVPPPYVMSRP, MSQTPYARPQYV, MTSNPHLNPGPR, MGHNINIPRTPL, LSTPLPYDMRRS, MTRIQDSPYDLR, MSTPPIREQAAH, MTNLPTVTQFPP, MTPIATSIPPQM, MTPTTPIPSLPQ, MTSPHPQTPNLT, MTQQPPLPHPAK, LAKPLPTTSNTG, LSKPIPHIPSSIG, CICRGVGCCLLL, LQPPSMITHPST, LTPPNQVLNPLY, AFPMVGGPDHFR, MLMPQPAHHNNS, AQAMANPLGSHI, SSRIPGFPDPLH, SMRGLPELNPRI, MSSPTVSSAPQY, VLSMQTPPTPLL, THAMSHLDKAH, MAVQPPNTSTSN, MAINDTYPPPRP, MMPPPTSLPSPS, LAQNPIYRAHPH, MQPRPQTLTPAS, LTVPVPVSFAVH, LTSPFSTPLNPR, MAGQPKDSSKTL, ANTPPHTILSTE, MGMTVPENLIVQ, and MTPIQSTQYPHS, or a derivative thereof, wherein said targeting peptide delivers the pharmaceutical agent for uptake by a target cell.

40. A method according to claim 39 wherein said targeting peptide comprises an amino acid sequence selected from the group consisting of SIGYPLP, NTGPNTV, LSNFHSS, GILSPSH MSSPGPA, MSLTTPPAVARP, MTPFPTSNEANL and MGMTVPENLIVQ, or a derivate thereof.

41. A method according to claim 39 wherein said targeting peptide comprises an amino acid sequence selected from the group consisting of SIGYPLP, NTGPNRV, LSNFHSS, GILSPSH and MSPPGPA or a derivative thereof.

42. A method according to claim 39 wherein said targeting peptide comprises an amino acid sequence selected from the group consisting of MSLTTPPAVARP, MTPFPTSNEANL and MGMTVPENLIVQ, or a derivative thereof.

43. A method according to claim 39 wherein said targeting peptide is up to 100 amino acids long.
